(19) United States
(12) Patent Application Publication
(10) Pub. No.: US 2005/0014954 A1
(43) Pub. Date: Jan. 20, 2005

(54) Pyrrole synthesis

(76) Inventors: Reinhold Ohrlein, Rehinfelden-Herten (DE); Gabriele Baisch, Binzen (DE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/496,282

(22) PCT Filed: Nov. 14, 2002
(86) PCT No.: PCT/EP02/12747

(30) Foreign Application Priority Data
Nov. 22, 2001 (EP) 01811125.2

(51) Int. Cl. C07D 207/30

(52) U.S. Cl. 548/560

(57) ABSTRACT

The invention relates to a novel process for the preparation of N-substituted pyrroles, especially of formula (V), wherein the radicals are as defined in the description, by intermolecular aza-Wittig reaction starting from organic azides and 1,4-dioxo compounds. The invention relates also to novel iminophosphorane intermediates for this synthesis. The resulting pyrroles are useful, for example, in the organic synthesis of pharmaceuticals or other active substances and chemicals.

### SUMMARY OF THE INVENTION

The invention relates to a novel process for the preparation of N-substituted pyrroles, especially of N- and 2-C- to 5-C-substituted pyrroles, by intermolecular aza-Wittig reaction starting from organic azides and 1,4-dioxo compounds. The invention relates also to novel iminophosphorane intermediates for that synthesis. The resulting pyrroles are useful, for example, in the organic synthesis of pharmaceuticals or other active substances.

### BACKGROUND TO THE INVENTION

Pyrrole ring systems not only are used industrially as constituents of various pigments, for example, but are also widespread in nature, e.g. as constituents of natural materials (see Comprehensive Heterocyclic Chemistry, A. R. Katritzky et al, Eds. CD-ROM (1997), CAN 127: 346376 AN 1997: 685558) or as constituents of pharmaceutical active ingredients, for example in atorvastatin (see WO 89/07598).

In a variant of the aza-Wittig reaction, so-called iminophosphoranes having a P═N double bond are generated from trialkyl-, triaryl-, trialkoxy- or triaryloxy-phosphorus compounds (phosphorus(III) compounds) and organic azides, with nitrogen being removed (see Y. G. Golobov, Tetrahedron 48(8), 1353 (1992) and S. Eguchi et al., Org. Prep. Proc. Int. 1992, 211). Such phosphorane imines can be isolated, but are usually immediately reacted (in situ) with an aldehyde or ketone, a C═N double bond being created analogously to the Wittig reaction. This extremely useful reaction has been used for the preparation of heterocycles, e.g. oxazoles, pyrazines, pyrazoles etc. (see H. Warmhoff et al., Advances in Heterocyclic Chemistry, Vol 64, Academic Press, New York 1995). Pyrroles have been obtained by this method solely by intramolecular reaction (see S. Eguchi et al., loc. cit.). In those cases, however, it is in particular not possible to obtain N-substituted pyrroles.

The aim of the invention is to provide a new process for the preparation of pyrroles that are N-substituted and especially additionally substituted at up to four of the carbon atoms, in the light of the fact that such compounds are otherwise obtainable on an industrial scale only with great difficulty, especially when all the ring atoms are to be in substituted form.

### GENERAL DESCRIPTION OF THE INVENTION

It has now been found, surprisingly, that pyrroles that are N-substituted and especially additionally substituted at up to four of the ring carbon atoms of the pyrrole can be obtained in high yield by intermolecular aza-Wittig reaction starting from organic azides and 1,4-dioxo compounds, especially 1,4-diketo compounds.

The deoxygenation of the dioxo compound is effected on the one hand by the phosphine reagent and on the other hand by water removal, the aromatic pyrrole system being synthesised in a single step.

The synthesis of the pyrrole is effected under Staudinger conditions (see H. Staudinger, E. Hauser, Helv. Chim. Acta 4, 861 (1921), H. Staudinger, J. Meyer, Helv. Chim. Acta 2, 635 (1919) or for a review see Y. G. Golobov et al., Tetrahedron 37, 437 (1981)), so that an N- and C-substituted pyrrole ring can be synthesised in one step.

The advantages of that ring-closure reaction include:
a) quasi reduction of the azide is effected in situ to form the reactive iminophosphorane;
b) the process is compatible with many different functionalities;
c) the reaction conditions of the process are extremely mild;
d) it is possible in particular to prepare sterically very bulky, highly substituted pyrroles which by other methods are obtainable only with difficulty and/or in a low yield (see J. A. Joule and G.-F. Smith, Heterocyclic Chemistry, R. van Norstrand, Wokingham, Berkshire (England) 1983, ISBN 0-442-30212-6);
e) using the process it is generally possible to prepare in one step N-substituted pyrroles which additionally carry up to four identical or, especially, different substituents at the pyrrole ring carbon atoms;
f) whereas amines are generally prepared by reduction of the corresponding azides, that step is omitted from the pyrrole synthesis described herein, which is advantageous from the safety standpoint.

### DETAILED DESCRIPTION OF THE INVENTION

The invention relates (I) especially to a process for the preparation of pyrroles of formula V 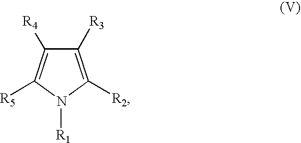
wherein R₁ is an organic substituent and
R₂, R₃, R₄ and R₅ are each independently of the others hydrogen or an inorganic or (preferably) organic substituent bonded by way of a carbon atom or hetero atom belonging to the radical, or a pair or pairs of those radicals may form a bridge bonded by way of carbon and/or hetero atoms,
wherein an iminophosphorane of formula IIa
   R₁—N═(PR^{x})₃ (IIa),
wherein
R₁ Is as defined for compounds of formula V and
R^{x} is unsubstituted or substituted alkyl, unsubstituted or substituted aryl, unsubstituted or substituted alkoxy or unsubstituted or substituted aryloxy,
is reacted with a dioxo compound of formula III 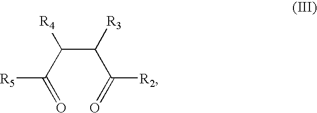
wherein the radicals R₂, R₃, R₄ and R₅ are as defined for compounds of formula V, in the presence of an acid;
functional groups in the starting materials being, if necessary, in protected form and any protecting groups being removed, if necessary, at suitable stages.

Preferably (ii) the iminophosphorane of formula IIa is obtained beforehand (especially in situ) by reaction of an azide of formula I
R₁—N₃ (I),
wherein R₁ is as defined for compounds of formula V, with a phosphorus(III) compound of formula II
P(R^{x})₃ (II),
wherein R^{x} is unsubstituted or substituted alkyl, unsubstituted or substituted aryl, unsubstituted or substituted alkoxy or unsubstituted or substituted aryloxy, it being possible in this case too for functional groups in the starting materials to be, if necessary, in protected form and for any protecting groups to be removed at suitable stages.

The invention relates (iii) also to a process for the preparation of atorvastatin, which comprises one or both of the afore-mentioned reactions (i) and (ii), the process including, if necessary, also the removal of protecting groups and/or cleavage of a lactone ring.

The invention relates also to iminophosphoranes of formula IIa wherein R₁ is a radical of sub-formula IA or sub-formula IB 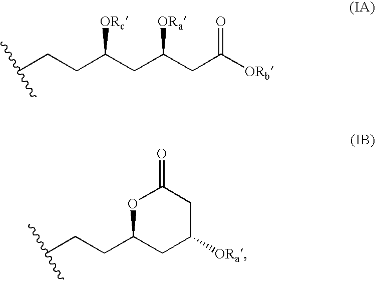
wherein Rₐ′ and R_{c}′ are each independently of the other hydrogen or a hydroxy-protecting group, or Rₐ′ and R_{c}′ together are a bridging hydroxy-protecting group; and R_{b}′ (only present in formula IA) is a carboxy-protecting group.

The general terms used hereinabove and hereinbelow (including the reactions and reaction conditions) preferably have the meanings given below, unless indicated otherwise—these specific definitions and reaction descriptions can be used, independently of one another, instead of the general terms mentioned hereinabove and hereinbelow, in each case resulting in preferred embodiments of the invention:

The adjective “lower” indicates that the radical in question has preferably up to 7 carbon atoms, especially up to 4 carbon atoms. Lower alkyl, for example, is preferably C₁-C₇alkyl, especially C₁-C₄alkyl, and may be unbranched or mono- or poly-branched, where possible. Unsaturated radicals, such as alkenyl or alkynyl, have at least two carbon atoms, preferably from 2 to 7, especially from 3 to 7, more especially 3 or 4.

An organic radical is preferably such a radical having from 1 to 50 carbon atoms (apart from cyano which here is included in the inorganic substituents), is saturated or unsaturated or partially unsaturated (in the latter cases preferably by inclusion of the multiple bonds in the aromatic systems), it also being possible for one or more (but not all) of the carbon atoms to be replaced by hetero atoms, especially those selected from the group comprising N (including NH), O, S (including S(═O) or S(═O)₂), Se and P, insofar as those radicals are chemically stable. The organic radical can be additionally substituted or unsubstituted.

An organic substituent is preferably unsubstituted or substituted alkyl, unsubstituted or substituted (especially C₂-C₇-)alkenyl having one or more double bonds, unsubstituted or substituted (especially C₂-C₇-)alkynyl having one or more triple bonds, unsubstituted or substituted cycloalkyl, unsubstituted or substituted aryl or unsubstituted or substituted heterocyclyl, or (preferably in the case of R₃ or R₄) is one of those radicals (especially unsubstituted or substituted alky) bonded by way of a bivalent radical C(═O)NH— or especially C(═O)O— belonging to the respective organic radical, the carbon atom of that linking bivalent radical being bonded to the pyrrole ring in formula V. As substituted alkyl R₁ special preference is given to a radical of sub-formula IA or of formula IB 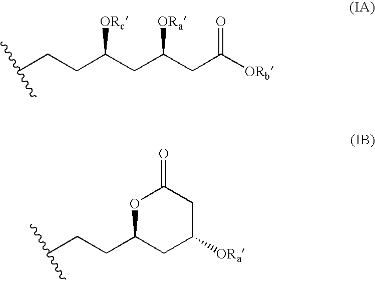
wherein Rₐ′ and R_{c}′ are each independently of the other hydrogen or a hydroxy-protecting group, or Rₐ′ and R_{c}′ together are a bridging hydroxy-protecting group; and R_{b}′ (only present in formula IA) is a carboxy-protecting group. Especially preferred as organic substituent R₁ is lower alkyl, e.g. hexyl, such as n-hexyl, or a radical of sub-formula IA or IB wherein R_{c}′ and Rₐ′ together are lower alkylidene, especially isopropylidene (1,1-dimethyl-methylene) and R_{b}′ is lower alkyl, especially ethyl or methyl, in each case where present.

An organic radical can also be bonded by way of a hetero atom, especially by way of nitrogen (including NH or NZ, wherein Z is a further organic radical, especially alkyl or substituted alkyl), sulfur (including S, S(═O) or S(═O)₂); or especially oxygen.

An inorganic radical is preferably cyano, or (especially for substituents R₃ and R₄) halogen, also mercapto, hydroxy, amino, hydrazino, hydroximino, sulfo, sulfamoyl or phosphono.

A bridge bonded by way of carbon and/or hetero atoms (the latter especially as defined above for organic radicals) that is formed from two of the radicals R₂, R₃, R₄ and R₅ is especially alkylenedioxy, such as lower alkylenedioxy, e.g. ethylenedioxy, or especially alkylene, more especially C₂-C₆alkylene, or the bridge forms together with the bonding carbon atoms a fused benzo ring which is unsubstituted or substituted (that is to say in the unsubstituted case the bridge has the formula —CH═CH—CH═CH—). The remaining radicals may likewise form a bridge or they may be the radicals otherwise mentioned for R₂, R₃, R₄ or R₅.

R₂ and R₅ are preferably organic substituents bonded by way of a carbon atom, preferably those described hereinabove and hereinbelow as being preferred, whereas R₃ and R₄ are hydrogen or an inorganic or organic substituent bonded by way of a carbon atom or hetero atom belonging to the radical, preferably as described above and below as being preferred.

“Substituted” in the case of radicals such as organic radicals, alkyl, aryl, cycloalkyl, heterocyclyl or fused benzo rings means especially that one or more, especially up to five, preferably up to three, hydrogen atoms of the radical in question have been replaced by the corresponding number of substituents, the substituents being selected independently of one another from the group consisting of alkyl, preferably lower alkyl, e.g. methyl, ethyl or propyl, halo-lower alkyl, such as fluoro-lower alkyl, e.g. trifluoromethyl, C₆-C₁₆aryl, preferably phenyl or naphthyl (C₆-C₁₆aryl, especially phenyl or naphthyl, being unsubstituted or substituted by one or more, especially up to three, substituents selected independently of one another from halogen, carboxy, lower alkoxycarbonyl, hydroxy, lower alkoxy, phenyl-lower alkoxy, lower alkanoyloxy, oxo (when present at a carbon or sulfur atom bonding to the rest of the molecule, a corresponding acyl radical is present), lower alkanoyl, amino, N-lower alkylamino, N,N-di-lower alkylamino, N-phenyl-lower alkylamino, N,N-bis(phenyl-lower alkyl)amino, lower alkanoylamino, fluoro-lower alkyl, such as trifluoromethyl, and sulfo), C₃-C₁₀cycloalkyl, hydroxy, lower alkoxy, e.g. methoxy, phenyl-lower alkoxy, lower alkanoyloxy, amino, N-lower alkylamino, N,N-di-lower alkylamino, N-phenyl-lower alkylamino, N,N-bis(phenyl-lower alkyl)amino, lower alkanoylamino, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, arylamino, especially phenylamino, carboxy, lower alkoxycarbonyl, phenyl-, naphthyl- or fluorenyl-lower alkoxycarbonyl, such as benzyloxycarbonyl, lower alkanoyl, sulfo, lower alkanesulfonyl, e.g. methanesulfonyl (CH₃—S(O)₂—), phosphono (—P(═O)(OH)₂), hydroxy-lower alkoxyphosphoryl or di-lower alkoxyphosphoryl, carbamoyl, mono- or di-lower alkyl-carbamoyl, sulfamoyl and mono- or di-lower alkylaminosulfonyl.

It will be clear to the person skilled in the art that such substituents can be present only at positions at which they are chemically possible and result in sufficiently stable chemical compounds, it being possible for the person skilled in the art to decide, on the basis of his or her expert knowledge or from simple routine experiments, which compounds fulfil those criteria. Tautomers are also included, for example in the case of keto-enol or imine-enamine tautomerism. The naming of the above-mentioned substituents therefore also includes their presence in forms modified by tautomerism.

Unsubstituted or substituted alkyl is preferably alkyl having up to 24 carbon atoms, especially C₁-C₁₂alkyl, preferably lower alkyl that is unsubstituted or substituted by one or more of the substituents mentioned above under “substituted”, it also being possible, in addition or alternatively, for unsubstituted or substituted aryl (especially as defined below), unsubstituted or substituted heterocyclyl (especially as defined below) and/or unsubstituted or substituted cycloalkyl (especially as defined below) to be present as further substituents. Preference is given to lower alkyl or arylaminocarbonyl (especially naphthyl- or more especially phenylaminocarbonyl).

Unsubstituted or substituted aryl preferably has a ring system containing not more than 24 carbon atoms, especially not more than 16 carbon atoms, is preferably mono-, bi- or tri-cyclic and is unsubstituted or is substituted, preferably as described under “substituted”. For example, aryl is selected from phenyl, naphthyl, indenyl, azulenyl and anthryl, preferably from unsubstituted or substituted phenyl or (especially 1- or 2-)naphthyl. Unsubstituted aryl (especially C₆-C₁₄aryl) or halo-substituted aryl (especially C₆-C₁₄aryl) is especially preferred.

Heterocyclyl is preferably a heterocyclic radical that is saturated or fully or partially unsaturated (multiple bonds preferably being in conjugated form, especially in aromatic systems) and is preferably a mono-, bi- or tri-cyclic ring system; has preferably from 3 to 24, especially from 4 to 16, ring atoms; one or more, especially from one to three, ring atoms being hetero atoms, especially selected from nitrogen, oxygen and sulfur, and heterocyclyl being unsubstituted or being substituted, especially as described under ‘substituted’. Examples of such heterocycles are imidazolyl, thienyl, furyl, tetrahydrofuryl, pyranyl, thiopyranyl, thianthrenyl, benzofuranyl, chromenyl, pyrrolyl, pyrrolidinyl, imidazolyl, imidazolidinyl, benzimidazolyl, pyrazolyl, pyrazinyl, pyrazolidinyl, pyranyl, thiazolyl, isothiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, indolyl, benzimidazolyl, coumaryl, indazolyl, triazolyl, purinyl, 4H-quinolizinyl, isoquinolyl, quinolyl, tetrahydroquinolyl, tetrahydroisoquinolyl, decahydroquinolyl, benzofuranyl, dibenzofuranyl, benzothiophenyl, dibenzothiophenyl, phthalazinyl, naphthyridinyl, quinoxalyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of those radicals being especially unsubstituted or mono- or poly-substituted, especially up to tri-substituted, by lower alkyl, such as methyl, or by lower alkoxy, such as methoxy.

Cycloalkyl is preferably C₃-C₁₀cycloalkyl, especially cyclopropyl, dimethylcyclopropyl, cyclobutyl, cyclopentyl, cyclohexyl or cycloheptyl, and is unsubstituted or, preferably, is substituted as described under ‘substituted’.

Unsubstituted or substituted alkoxy is unsubstituted or substituted alkyl, as defined above, that is bonded to the rest of the molecule by way of an oxygen atom, preferably an oxygen atom bonded terminally to the alkyl radical. Preference is given to lower alkoxy that is substituted, as described above under ‘substituted’, or especially is unsubstituted.

Unsubstituted or substituted aryloxy is unsubstituted or substituted aryl, as defined above, that is bonded to the rest of the molecule by way of an oxygen atom. Preference is given to phenyloxy that is substituted, as described above under ‘substituted’, or especially is unsubstituted.

R₂, R₃, R₄ and R₅ (in compounds of formulae III and V) are preferably selected from alkyl, especially lower alkyl, such as hexyl, e.g. n-hexyl, or isopropyl; aryl, especially phenyl or naphthyl; substituted aryl, such as halo-phenyl or halo-naphthyl, e.g. fluorophenyl; lower alkoxycarbonyl, especially ethoxy- or tert-butoxy-carbonyl; and arylaminocarbonyl, especially phenylaminocarbonyl (=N-phenyl-carbamoyl).

Preferably R₂ is lower alkyl, especially isopropyl; R₃ is arylaminocarbonyl, especially phenylaminocarbonyl; R₄ is aryl, especially phenyl; and R₅ is substituted aryl, especially fluorophenyl, more especially 4-fluorophenyl.

Halogen is especially fluorine, chlorine, bromine or iodine, especially chlorine or bromine.

In the processes mentioned hereinabove and hereinbelow, in the context of protecting functional groups in the compounds of formulae I to V in question that are not to participate in the reaction or would interfere with the reaction, such as hydroxy, carboxy, mercapto or amino, it is possible at any stage, even where not explicitly mentioned, for those functional groups to be converted into protected groups by the introduction of suitable protecting groups (especially hydroxy-protecting groups and/or carboxy-protecting groups), and/or at suitable stages, especially in the case of the end products, it is possible for one, some or all of the protecting groups present to be removed.

The protection of functional groups by such protecting groups, suitable reagents for their introduction, suitable protecting groups and reactions for their removal will be familiar to the person skilled in the art. Examples of suitable protecting groups can be found in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/I, Georg Thieme Verlag, Stuttgart 1974, in H.-D. Jakubke and H. Jescheit, “Aminosäuren, Peptide, Proteine”, Verlag Chemie, Weinheim, Deerfield Beach, and Basel 1982, and/or in Jochen Lehmann, “Chemie der Kohlenhydrate: Monosaccharide und Derivate”, Georg Thieme Verlag, Stuttgart 1974.

Suitable hydroxy-protecting groups are especially selected from those of the acyl or ester type, e.g. lower alkanoyl, such as formyl, acetyl or isobutyryl, benzoylformyl, chloroacetyl, dichloroacetyl, trichloroacetyl, trifluoroacetyl, methoxyacetyl, phenoxyacetyl, phenylacetyl, p-phenylacetyl, diphenylacetyl, 2,6-dichloro-4-methylphenoxyacetyl, 2,6-dichloro-4-(1,1,3,3-tetramethylbutyl)phenoxyacetyl, 2,4-bis(1,1-dimethylpropyl)phenoxyacetyl, chlorodiphenylacetyl, 3-phenylpropionyl, 4-azidobutyryl, 4-methylthiomethoxybutyryl, (E)-2-methyl-2-butenoyl, 4-nitro-4-methylpentanoyl, 4-pentenoyl, 4-oxopentanoyl, 4,4-(ethylenedithio)pentanoyl, 5-[3-bis(4-methoxyphenyl)hydroxymethylphenoxy)laevulinyl, pivaloyl, crotonoyl, monosuccinoyl, benzoyl, p-phenylbenzoyl, 2,4,6-trimethylbenzoyl, 2-(methylthiomethoxymethyl)benzoyl, 2-(chloroacetoxymethyl)benzoyl, 2-[(2-chloroacetoxy)ethyl]benzoyl, 2-[(2-benzyloxy)ethyl]benzoyl, 2-[2-(4-methoxybenzyloxy)ethyl]benzoyl, 2-iodobenzoyl, o-(dibromomethyl)benzoyl, o-(methoxycarbonyl)benzoyl, 2-chlorobenzoyl, 4-bromobenzoyl, 4-nitrobenzoyl, alkoxycarbonyl, such as methoxycarbonyl, ethoxycarbonyl, isobutoxycarbonyl, methoxymethylcarbonyl, 9-fluorenylmethoxycarbonyl, 2,2,2-trichloroethoxycarbonyl, 1,1-dimethyl-2,2,2-trichloroethoxycarbonyl, 2-(trimethylsilyl)ethoxycarbonyl, 2-(phenylsulfonyl)ethoxycarbonyl, 2-(triphenylphosphonio)ethoxycarbonyl, vinyloxycarbonyl, allyloxycarbonyl, p-nitrophenoxycarbonyl, benzyloxycarbonyl, p-methoxybenzyloxycarbonyl, 3,4-dimethoxybenzyloxycarbonyl, o-nitrobenzyloxycarbonyl, p-nitrobenzyloxycarbonyl, dansylethoxycarbonyl, 2-(4-nitrophenyl)ethoxycarbonyl, 2-(2,4-dinitrophenyl)ethoxycarbonyl, 2-cyano-1-phenylethoxycarbonyl, S-benzylthiocarbonyl, 4-ethoxy-1-naphthyloxycarbonyl, 3′,5′-dimethoxybenzoinyloxycarbonyl, 2-methylthiomethoxyethoxycarbonyl, N-phenylcarbamoyl, dimethylethylphosphinothiolyl, methyldithiocarbonyl; N,N,N′,N′-tetramethylphosphorodiamidoyl, sulfonyl, methanesulfonyl, benzenesulfonyl, toluenesulfonyl, 2-[(4-nitrophenyl)ethyl]sulfonyl, allylsulfonyl, 2-formylbenzenesulfonyl, nitroxy, or protecting groups of the ether type, such as methyl, substituted methyl, preferably lower alkoxymethyl, especially methoxymethyl (MOM), methylthiomethyl, (phenyldimethylsilyl)methoxymethyl, benzyloxymethyl, p-methoxybenzyloxymethyl, p-nitrobenzyloxymethyl, guaiacolmethyl, tert-butoxymethyl, 4-pentenyloxymethyl, silyloxymethyl, lower alkoxy-lower alkoxymethyl, especially 2-methoxyethoxymethyl (MEM), 2,2,2-trichloroethoxymethyl, 2-(trimethylsilyl)-ethoxymethyl or menthoxymethyl, tetrahydropyranyl, 3-bromotetrahydropyranyl, tetrahydrothiopyranyl, 4-methoxythiopyranyl, 1-methoxycyclohexyl, 4-methoxytetrahydrothiopyranyl, S,S-dioxy-4-methoxytetrahydrothiopyranyl, 1-[(2-chloro-4-methyl)phenyl]-4-methoxypiperidin-4-yl, 1-(2-fluorophenyl)-4-methoxypiperidin-4-yl, 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl; substituted ethyl, such as 1-ethoxyethyl, 1-(2-chloroethoxy)ethyl, 1-[2-(trimethylsilyl)ethoxy]ethyl, 1-methyl-1-methoxyethyl, 1-methyl-1-benzyloxyethyl, 1-methyl-1-benzyloxy-2-fluoroethyl, 1-methyl-1-phenoxyethyl, 2,2,2-trichloroethyl, 1,1-dianisyl-2,2,2-trichloroethyl, 1,1,1,3,3,3-hexafluoro-2-phenylisopropyl, 2-trimethylsilylethyl, 2-(benzylthio)ethyl, 2-(phenylselenyl)ethyl, tert-butyl; allyl or propargyl, substituted phenyl ethers, such as p-chlorophenyl, p-methoxyphenyl, p-nitrophenyl, 2,4-dinitrophenyl or 2,3,5,6-tetrafluoro-4-(trifluoromethyl)phenyl, benzyl, substituted benzyl, such as p-methoxybenzyl, 3,4-dimethoxybenzyl, o-nitrobenzyl, p-nitrobenzyl, p-halobenzyl, e.g. p-bromobenzyl, 2,6-dichlorobenzyl, p-cyanobenzyl, p-phenylbenzyl, 2,6-difluorobenzyl, p-azidobenzyl, 4-azido-3-chlorobenzyl, 2-trifluoromethylbenzyl or p-(methylsulfinyl)benzyl, 2- or 4-picolyl, 3-methyl-2-picolyl, 2-quinolinylmethyl, 1-pyrenylmethyl, diphenylmethyl, p,p′-dinitrobenzhydryl, 5-dibenzosuberyl, triphenylmethyl, α-naphthyidiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl), 4,4′,4″-tris(laevulinoyloxyphenyl)methyl, 4,4′,4″-tris(benzoyloxyphenyl)methyl, 4,4′-dimethoxy-3″-[N-(imidazolylmethyl)]trityl, 4,4′-dimethoxy-3″-[N-(imidazolylethyl)carbamoyl]trityl, 1,1-bis(4-methoxyphenyl)-1′-pyrenylmethyl, 4-(17-tetrahydrobenzo[a,c,g,i]fluorenylmethyl)-4′,4″-dimethoxytrityl, 9-anthryl, 9-(9-phenyl)xanthenyl, 9-(9-phenyl-10-oxo)anthryl, 1,3-benzodithiolan-2-yl, S,S-dioxo-benzoisothiazolyl; of the silyl ether type, such as tri-lower alkylsilyl, e.g. trimethylsilyl, triethylsilyl, triisopropylsilyl, dimethylisopropylsilyl, diethylisopropylsilyl, dimethylthexylsilyl, tert-butyldimethylsilyl or di-tert-butylmethylsilyl, tert-butyldiphenylsilyl, triphenylsilyl, diphenylmethylsilyl, tris(trimethylsilyl)silyl, (2-hydroxystyryl)dimethylsilyl, (2-hydroxystyryl)diisopropylsilyl, tert-butylmethoxyphenylsilyl or tert-butoxydiphenylsilyl.

Bridging protecting groups can likewise be used where a molecule contains two hydroxy groups (for example bridging hydroxy-protecting groups formed by Rₐ and R_{c} or Rₐ′ and R_{c}′ together) or a hydroxy-protecting group and a carboxy group (for example bridging protecting groups formed by Rₐ and R_{b} or Rₐ′ and R_{b} in the molecules of the corresponding formulae mentioned hereinabove and hereinbelow in which those radicals are present).

A bridging hydroxy-protecting group (especially one formed by Rₐ′ and R_{c}′) is preferably selected from methylene, ethylidene, tert-butylmethylidene, 1-tert-butylethylidene, 1-phenylethylidene, 1-(4-methoxyphenyl)ethylidene, 2,2,2-trichloroethylidene, vinylmethylidene, cyclopentylidene, cyclohexylidene, cycloheptylidene, benzylidene, p-methoxybenzylidene, 2,4-dimethoxybenzylidene, 3,4-dimethoxybenzylidene, 2-nitrobenzylidene, 4-nitrobenzylidene, mesitylene, phenyl-(1,2-bis(methylenyl)), methoxymethylene, ethoxymethylene, dialkylsilylene, such as tert-butylsilylene, 1,3-(1,1,3,3-tetraisopropyldisiloxanylidene), 1,1,3,3-tetra-tert-butoxydisiloxanylidene, —C(═O) or especially isopropylidene.

Carboxy-protecting groups are especially ester-forming, enzymatically and/or chemically removable protecting groups, preferably enzymatically and/or chemically removable protecting groups, such as heptyl, 2-N-(morpholino)ethyl, cholinyl, methoxyethoxyethyl or methoxyethyl; or those which are primarily chemically removable, e.g. alkyl, such as lower alkyl, especially methyl, ethyl, substituted lower alkyl (except for benzyl and substituted benzyl), such as substituted methyl, especially 9-fluorenylmethyl, methoxymethyl, methoxyethoxymethyl, methylthiomethyl, 2-(trimethylsilyl)ethoxymethyl, benzyloxymethyl, pivaloyloxymethyl, phenylacetoxymethyl, triisopropylsilylmethyl, 1,3-dithianyl-2-methyl, dicyclopropylmethyl, acetonyl, phenacyl, p-bromophenacyl, α-methylphenacyl, p-methoxyphenacyl, desyl, carbamidomethyl, p-azobenzenecarboxamidomethyl, N-phthalimidomethyl or 4-picolyl, 2-substituted ethyl, 2,2,2-trichloroethyl, 2-(trimethylsilyl)ethyl, 2-methylthioethyl, 2-(p-nitrophenylsulfenyl)ethyl, 2-(p-toluenesulfonyl)ethyl, 2-(2′-pyridyl)ethyl, 2-(p-methoxyphenyl)ethyl, 2-(diphenylphosphino)ethyl, 1-methyl-1-phenylethyl, 2-(4-acetyl-2-nitrophenyl)ethyl or 2-cyanoethyl, tert-butyl, 3-methyl-3-pentyl, 2,4-dimethyl-3-pentyl or ω-chloro-lower alkyl, especially 5-chloropentyl, cyclopentyl, cyclohexyl, lower alkenyl, especially allyl, methallyl, 2-methylbut-3-en-2-yl, 3-methylbut-2-enyl or 3-buten-1-yl, substituted lower alkenyl, especially 4-(trimethylsilyl)-2-buten-1-yl, cinnamyl or α-methylcinnamyl, lower alkynyl, such as prop-2-ynyl, phenyl, substituted phenyl, especially 2,6-dialkylphenyl, such as 2,6-dimethylphenyl, 2,6-diisopropylphenyl, 2,6-di-tert-butyl-4-methylphenyl, 2,6-di-tert-butyl-4-methoxyphenyl, p-(methylthio)phenyl or pentafluorophenyl, benzyl, substituted benzyl, especially triphenylmethyl, diphenylmethyl, bis(o-nitrophenyl)methyl, 9-anthrylmethyl, 2-(9,10-dioxo)anthrylmethyl, 5-dibenzosuberyl, 1-pyrenylmethyl, 2-(trifluoromethyl)-6-chromonylmethyl, 2,4,6-trimethylbenzyl, p-bromobenzyl, o-nitrobenzyl, p-nitrobenzyl, p-methoxybenzyl, 2,6-dimethoxybenzyl, 4-(methylsulfinyl)benzyl, 4-sulfobenzyl, 4-azidomethoxybenzyl, 4-(N-[1-(4,4-dimethyl-2,6-dioxocyclohexylidene)-3-methylbutyl]amino)benzyl, piperonyl or p-polymer-benzyl, tetrahydropyranyl, tetrahydrofuranyl, or silyl radicals, such as tri-lower alkylsilyl, especially trimethylsilyl, triethylsilyl, tert-butyidimethylsilyl, isopropyldimethylsilyl or di-tert-butylmethylsilyl, or phenyl-di-lower alkylsilyl, such as phenyldimethylsilyl; alternatively a carboxy group can also be protected in the form of an oxazolyl, 2-alkyl-1,3-oxazolinyl, 4-alkyl-5-oxo-1,3-oxazolidinyl or 2,2-bistrifluoromethyl-4-alkyl-5-oxo-1,3-oxazolidinyl radical. Amide-protecting groups are especially allyl, tert-butyl, N-methoxy, N-benzoyloxy, N-methylthio, triphenylmethylthio, tert-butyldimethylsilyl, triisopropylsilyl, 4-(methoxymethoxy)phenyl, 2-methoxy-1-naphthyl, 9-fluorenyl, tert-butoxycarbonyl, N-benzyloxycarbonyl, N-methoxy- or N-ethoxy-carbonyl, toluenesulfonyl, N-buten-1-yl, 2-methoxycarbonylvinyl, or especially alkyl, such as lower alkyl, or more especially substituted alkyl, especially benzyl, benzyl substituted by one or more radicals selected from lower alkoxy, such as methoxy, lower alkanoyloxy, such as acetoxy, lower alkylsulfinyl, such as methylsulfinyl, dicyclopropylmethyl, methoxymethyl, methylthiomethyl and N-benzoyloxymethyl; or bis(trimethylsilyl)methyl, trichloroethoxymethyl, tert-butyldimethylsilyloxymethyl, pivaloyloxymethyl, cyanomethyl, benzyl, 4-methoxybenzyl, 2,4-dimethoxybenzyl, 3,4-dimethoxybenzyl, 2-acetoxy-4-methoxybenzyl, o-nitrobenzyl, bis(4-methoxyphenyl)phenylmethyl, bis(4-methylsulfinylphenyl)methyl, pyrrolidinomethyl, diethoxymethyl, 1-methoxy-2,2-dimethylpropyl or 2-(4-methylsulfonyl)ethyl.

A protecting group function can also be provided by the intramolecular formation of lactones (by reaction of a hydroxy function with a carboxy function), the lactone cleavage being effected under customary conditions, for example analogously to the cleavage of carboxy groups protected in ester form.

It is characteristic of protecting groups that they are simple to remove without undesirable secondary reactions taking place, for example by solvolysis, reduction, photolysis or alternatively under conditions analogous to physiological conditions, for example enzymatically.

The person skilled in the art will know which protecting groups, methods for their introduction and methods for their removal can be used for which reactions and compounds.

The reaction of the iminophosphorane of formula IIa with the dioxo compound of formula III is preferably carried out under the following conditions:

The iminophosphorane IIa (for example isolated after being prepared beforehand and if desired after being stored, or generally without working-up with immediate further use in situ) is reacted by combining the iminophosphorane, preferably an iminophosphorane solution, with a mixture of a 1,4-dioxo compound III and an acid in one of the solvents listed below, for example by adding the iminophosphorane solution to the dioxo compound of formula III and the acid.

The acid used is preferably a moderately acidic ion exchanger, a moderately acidic inorganic acid, such as phosphoric acid, or an organic acid, e.g. an organic phosphoric acid derivative or a carboxylic acid, or a mixture of such acids. Special preference is given to sterically hindered aliphatic or aromatic carboxylic acids, such as 2-methylbutyric acid or especially α,α-di-lower alkyl-lower alkanecarboxylic acids, such as pivalic acid, or more especially polyalkylated, especially 2,(4),6-di(or tri)-alkylated benzoic acids, such as 2,4,6-trimethylbenzoic acid, 2,4,6-triisopropylbenzoic acid or 2,4,6-tri-tert-butylbenzoic acid, or mixtures of two or more of those acids. Those acids may advantageously also be bonded (especially covalently) to a polymeric carrier.

The solvent used is an organic solvent, preferably a dry aprotic organic solvent, especially an ether, preferably a di-lower alkyl ether, such as diethyl ether or methyl tert-butyl ether, or a cyclic ether, such as tetrahydrofuran or dioxane, an aliphatic or aromatic hydrocarbon, such as benzene, toluene or xylene, a halogenated hydrocarbon, such as methylene chloride, or the like, or mixtures of two or more such solvents.

The reaction of the phosphorane imine IIa with the 1,4-dioxo compound III can be carried out in the presence of further reagents that bind the water of reaction formed, such as hygroscopic salts, e.g. calcium chloride, magnesium sulfate or sodium sulfate, “diphosphorus pentoxide” (free or bonded to inert carriers), silica gel or aluminium oxide—organic orthoesters, such as ortho-acetic acid ethyl ester or molecular sieves (for example molecular sieve 3A or 4A) have proved especially advantageous.

The molar ratio of phosphorane imine IIa to dioxo compound III and acid IV is preferably from about 1 to 1.5:1, especially 1:1.

The mixture is preferably stirred at temperatures of from room temperature to 110° C., especially from 40° C. to 70° C., until the component used in a less than stoichiometric amount has been consumed.

Preferably after customary working-up, for example extractive and/or chromatographic working-up, the pure pyrroles V are obtained.

The preparation of the iminophosphorane of formula IIa from an azide of formula I by reaction with a phosphorus(III) compound of formula II is preferably effected under the following conditions:

The azide of formula I is reacted in a dry organic solvent, as defined above for the reaction between compounds of formulae IIa and III, at preferred temperatures of from −20° C. to the reflux temperature, especially from room temperature to 40° C., with a suitable amount, especially from 1 to 1.5 equivalents, preferably from 1 to 1.1 equivalents, of the compound of formula II—the compound of formula I especially being used as initial charge and the compound of formula II being added thereto. The reaction is preferably carried out until the component used in a less than stoichiometric amount has reacted completely. The phosphorus(III) compound of formula II can advantageously also be used bound to a polymeric carrier (for example based on polystyrene, see also: Hemming et al., Synlett 11, 1565 (2000)).

Preferred Embodiments of the Invention:

Preferred embodiments of the invention are obtained by using the above-mentioned more specific meanings in place of more general terms and reaction conditions in the more general definitions, it being possible to replace one, some or all of the more general terms by more specific meanings, in each case resulting in preferred embodiments of the invention.

Preferred embodiments of the invention can be found especially in the claims which are included herein by reference, it being possible also in the claims for more general terms to be defined by the more specific terms mentioned hereinabove and hereinbelow.

Special preference is given to the starting compounds and final compounds mentioned in the Examples, and to the reaction conditions and/or reagents mentioned therein.

Preparation of the Starting Materials:

An extremely wide variety of known synthesis methods is available for the preparation of the starting compounds (see e.g. for the preparation of primary azides “The Azido-Group”, in: Patal-series 1971, written by M. E. C. Biffin, J. Miller and D. B. Paul, Interscience Publishers, London, and for the preparation e.g. of diketones see M. Yasuda et al., J. Org. Chem. 62, 8282 (1997) and literature cited therein, and also WO 89/07598).

A compound of formula IA falling within the scope of formula I 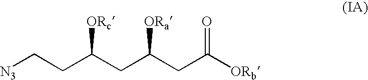
wherein Rₐ′ and R_{c}′ are each independently of the other hydrogen or a hydroxy-protecting group, or Rₐ′ and R_{c}′ together are a bridging hydroxy-protecting group; and R_{b}′ is a carboxy-protecting group (especially (3R,5R)-7-azido-3,5-dihydroxy-heptanoic acid ethyl ester or (3R,5R)-7-azido-3,5-(2′,2′-isopropylidene-dioxy)heptanoic acid ethyl ester) is preferably obtained as follows:

The process according to the invention starts from the key intermediate of formula VI 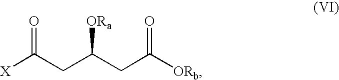
wherein X is halogen, Rₐ is a hydroxy-protecting group and R_{b} is a carboxy-protecting group, which is ethenylated as described below:

The ethenylation is carried out with an ethylene of formula VII 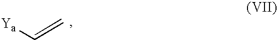
wherein Yₐ is halogen or hydrogen, yielding a keto compound of formula VIII 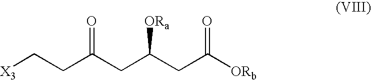
wherein Xₐ is halogen, Rₐ is hydrogen (obtainable after selective removal of a hydroxy-protecting group Rₐ) or is a hydroxy-protecting group and R_{b} is a carboxy-protecting group; then either the compound of formula VII is reacted further by reacting it with a salt of hydrazoic acid to form an azido compound of formula IX 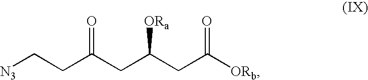
wherein Rₐ is hydrogen or a hydroxy-protecting group and R_{b} is a carboxy-protecting group. The compound of formula IX (when Rₐ is a hydroxy-protecting group, after prior selective removal thereof) is then reduced diastereoselectively by means of a suitable reagent to form the syn-diol compound of formula IA wherein Rₐ′ is hydrogen and R_{c}′ is hydrogen; or, after subsequent introduction of protecting groups, Rₐ′ and R_{c}′ are each independently of the other hydrogen or a protecting group, with the proviso that at least one of the two radicals is a protecting group, or Rₐ′ and R_{c}′ together are a bridging hydroxy-protecting group; and R_{b}′ is a carboxy-protecting group, and, in a case where the introduction of a bridging hydroxy-protecting group is desirable, when Rₐ′ and R_{c}′ are each hydrogen, the bridging hydroxy-protecting group formed by Rₐ′ and R_{c}′ together can be introduced using a suitable reagent;
or (when hydroxy-protecting groups Rₐ are present, after removal thereof) the compound of formula VII is first converted diastereoselectively into a syn-diol compound of formula IX* 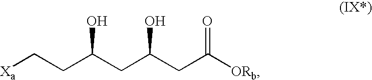
wherein Xₐ is halogen and R_{b} is a carboxy-protecting group, which compound is then converted by reaction with a salt of hydrazoic acid (if necessary after the introduction of hydroxy-protecting groups, as described for compounds of formula IX) into the compound of formula IA.

The compound of formula (VI) is preferably prepared starting from a compound of formula X 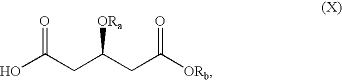
wherein R_{b} is a hydroxy-protecting group (or, less preferred, because the enantiomeric excess=ee is then lower, also hydrogen) and R_{b} is a carboxy-protecting group, which compound is reacted with a reagent that introduces the radical X.

The compound of formula X in turn is advantageously prepared by hydrolysing a compound of formula XI 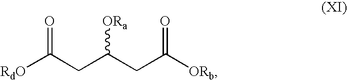
wherein Rₐ is a hydroxy-protecting group (or, less preferred, because the ee is then lower, also hydrogen), R_{b} is a carboxy-protecting group and R_{d} is hydrocarbyl, by means of an enantioselective catalyst (preferably by hydrolysis by means of a biocatalyst) with removal of the radical R_{d}, the corresponding compound of formula X being obtained directly.

The compound of formula XI is advantageously obtained by reacting a glutaric acid derivative of formula XI 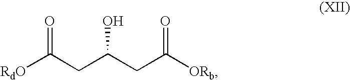
wherein R_{b} and R_{d} are as defined for compounds of formula XI, by introduction of a hydroxy-protecting group with the corresponding reagent suitable for the introduction of protecting groups.

Compounds of formula XII are known, can be prepared according to methods known per se or are commercially available.

The reaction of the intermediate of formula VI with an ethylene of formula VII is effected preferably in the presence of a Lewis acid, such as FeCl₃, SbCl₅, SnCl₄, BF₃, TiCl₄, ZnCl₂ or especially aluminium chloride (AlCl₃), preferably in a suitable solvent, especially a halogenated hydrocarbon, such as chloroform, methylene chloride or ethylene chloride, at preferred temperatures of from −10° C. to the reflux temperature, especially from 0 to 30° C.

Any hydroxy-protecting groups Rₐ can then, if necessary, be removed selectively from the compound of formula VIII by customary methods, especially by the methods described in the standard works mentioned above.

“Selectively” means especially enzymatically. In particular, lower alkanoyl, such as acetyl, is removed enzymatically, for example by esterases, such as pig liver esterase, in suitable buffers, such as phosphate buffer, at preferred pH values of from 5 to 9, especially from 6 to 8. Further possible enzymes and reaction conditions will be found below under the definition of biocatalysts for the hydrolysis. Lower alkoxymethyl, such as MOM, or lower alkoxy-lower alkoxymethyl, such as MEM, is removed by chemical standard methods.

The conversion of a compound of formula VII into a compound of formula IX, as defined above, using a salt of hydrazoic acid is preferably carried out with such a salt in the presence of a complex-forming agent for the metal cation, especially with an alkali metal azide, such as sodium or potassium azide, (in the absence or in the presence of a crown ether, especially 18-crown-6-ether) in a suitable solvent, preferably an aprotic solvent, such as a di-lower alkyl-lower alkanoylamide, e.g. dimethylformamide or dimethylacetamide, or a di-lower alkyl sulfoxide, e.g. dimethyl sulfoxide, or the like. The reaction can alternatively be carried out under conditions of phase transfer catalysis, i.e. in the presence of two-phase systems, such as water/organic solvent (such as halogenated hydrocarbons, e.g. methylene chloride, chloroform or dichloroethane), in the presence of lipophilic quaternary ammonium salts, such as hydrogen sulfate or chloride, e.g. tetrabutylammonium hydrogen sulfate, Aliquat 336, Adogen 464 (both consisting primarily of methyltrioctylammonium chloride), preferably tetra-lower alkylammonium bromide or iodide, such as tetrabutylammonium bromide or iodide or the like, the base being present in the aqueous phase.

The diastereoselective reduction of the obtainable azido compound of formula IX (if necessary after removal of the hydroxy-protecting group Rₐ, preferably as described above for the removal of the hydroxy-protecting group Rₐ from a compound of formula VIII) to form a compound of formula IA, as defined above, is then preferably carried out in a chelate-controlled manner, there being used as chelate-forming agent preferably a di-lower alkyl borinic acid lower alkyl ester, especially diethyl borinic acid ethyl ester. The reduction of the chelated 0]-hydroxyketone of formula IX is then effected with a complex hydride, preferably with an alkali metal borohydride, especially with sodium borohydride. As solvent there are preferably used ethers, such as cyclic ethers, especially tetrahydrofuran, and/or alcohols, such as lower alkanols, e.g. methanol, the preferred reaction temperatures being from 40 to −30° C., especially from −78 to −40° C. In a broader embodiment of the invention it is also possible to use alternative reducing agents, such as sodium cyanoborohydride, but this results in lower diastereoselectivity and is therefore less preferred.

Mutatis mutandis, the reaction conditions mentioned above for the preparation of the compound of formula IX and the subsequent diastereoselective reduction apply also to the conversion first by way of the compound of formula IX* by diastereoselective reduction of the compound of formula VIII and subsequent introduction of the azido group by replacement of Xₐ in a compound of formula IX*.

When it is desirable or necessary subsequently to introduce a protecting group into the compound of formula IA (Rₐ′, R_{c}′ or Rₐ′ and R_{c}′ as protecting group, especially Rₐ′ and R_{c}′ together as a bridging protecting group), this is carried out under standard conditions, preferably as described in the above-mentioned standard works.

Hydrocarbyl R_{d} in a compound of formula XI is preferably a saturated, fully or partially unsaturated, cyclic (having one or more, especially up to three, fused rings), linear, branched or mixed cyclic-linear or cyclic-branched hydrocarbon radical having up to 24 carbon atoms, preferably up to 10 carbon atoms, especially lower alkyl, and is unsubstituted or mono- or poly-substituted, preferably up to tri-substituted, especially by hydroxy, lower alkoxy, phenyl-lower alkoxy, lower alkanoyloxy, phenyl-lower alkanoyloxy, benzoyloxy, halogen, carboxy, lower alkoxycarbonyl or halo-lower alkyl, such as trifluoromethyl. Preference is given to lower alkyl, especially methyl or more especially ethyl, or lower alkoxy-lower alkyl, especially methoxymethyl. Preferably, in the compounds of formulae XI and XII the carboxy-protecting group R_{b} is identical to the hydrocarbyl group R_{d}, and is especially in each case lower alkyl, more especially methyl or ethyl, branched lower alkyl or lower alkoxy-lower alkyl, especially methoxymethyl.

The preparation of a compound of formula X is preferably effected with removal of the hydrocarbyl radical R_{d} in the presence of an enantioselective catalyst, especially a biocatalyst.

As biocatalysts for the hydrolysis there are suitable cells or ruptured cells with the enzymes mentioned below, or especially enzymes as such, preferably esterases, lipases and proteases (peptidases or amidases, see U. T. Bornscheuer and R. T. Kazlauskas, in: Hydrolases in Organic Synthesis, Wiley-VCH, 1999, pages 65-195, ISBN 3-527-30104-6). Common representatives of those classes of enzyme are especially animal esterases (e.g. pig liver esterase=PLE, pig pancreas esterase=PPL), esterases from microorganisms or fungi (B. subtilis esterase, Pichia esterases, yeast esterases, Rhizopus sp. esterases (RML, ROL), Penicillium sp. esterases, G. candidum (GCL), H. lanuginosa (HLL), Candida sp. (CAL-A, CAL-B, CCL), Aspergillus sp. (ANL), Pseudomonas sp. (PCL, PFL) and the like), and also proteases, e.g. subtilisin, thermitase, chymotrypsin, thermolysin, papain, aminoacylases, penicillin amidases, trypsin or the like, to name only a few. The person skilled in the art will be familiar with further suitable enzymes, and the enzymes that can be used are not limited to those mentioned in the above list. The enzymes can be obtained in the form of crude isolates and/or in purified form from natural sources and/or from recombinant microorganisms by means of modern cloning procedures via overexpression, amplification or the like. Commercially available enzymes are especially preferred. The enzymes can be present as such or immobilised or adsorbed on carriers, for example on silica gel, kieselguhr, such as Celite®, Eupergit® (Röhm & Haas, Darmstadt, Germany) or the like, or used in the form of “CLECs” (cross-linked enzymes), such as are available from ALTUS BIOLOGICS, the scope for use extending beyond the list given, as the person skilled in the art will know (see U. T. Bornscheuer and R. T. Kazlauskas, in: Hydrolases in Organic Synthesis, Wiley-VCH, 1999, pages 61-64, ISBN 3-527-30104-6; K. Faber in: Biotransformation in Organic Chemistry, Springer 1997, Third Edition, pages 345-357, ISBN 3-540-61688-8; H. J. Rehm, G. Reed in: Biotechnology, VCH 1998, Second Edition, pages 407-411). The enzymes can be used in pure organic solvents, e.g. liquid hydrocarbons, such as hexane, toluene or benzene, liquid ethers, such as diethyl ether, methyl tert-butyl ether or tetrahydrofuran, liquid halogenated hydrocarbons, such as methylene chloride, water or aqueous buffer solutions, in mixtures of those solvents, for example mixtures of one or more thereof with water or aqueous buffer solutions. The aqueous solution is preferably buffered, pH 5-9, it being possible to use customary buffer systems (see e.g. K. Faber in: Biotransformation in Organic Chemistry, Springer 1997, Third Edition, p. 305; or U. T. Bornscheuer and R. T. Kazlauskas, in: Hydrolases in Organic Synthesis, Wiley-VCH, 1999, pages 61-65). The pH is preferably kept substantially constant during the reaction. Most suitable for this purpose is an automatic titrator having a standardised acid or base solution, or manual titration. The reaction temperature is preferably in the range from 10 to 50° C., especially from 25 to 40° C. The amount of biocatalyst used and the concentrations of the reagents can be dependent upon the substrate and the reaction conditions (temperature, solvent etc.) selected in each case, as will be known to the person skilled in the art. There are preferably used commercially available enzymes (for example from Fluka, Sigma, Novo Nordisk, Amano, Roche and the like) or those listed in the current literature (see e.g. H.-J. Rehm, G. Reed in: Biotechnology, VCH 1998, 2^{nd} Edition, pages 40-42). Especially preferred for the preparation of enantiomerically pure compounds is α-chymotrypsin in phosphate buffer, especially at pH 7.0.

The preparation of a compound of formula XI from the free hydroxy compound of formula XII is effected with introduction of a hydroxy-protecting group, reagents that introduce suitable hydroxy-protecting groups being known, preferably as described in the mentioned standard works relating to protecting groups. The introduction of lower alkanoyl or lower alkoxy-lower alkanoyl is preferably carried out with a corresponding anhydride, especially a lower alkanoyl anhydride, such as acetic anhydride, or a corresponding acid halide, such as a lower alkoxy-lower alkanoyl halide, such as methoxyacetyl chloride, in the presence of a nitrogen base, especially pyridine, in the presence or absence of an inert solvent, especially a halogenated hydrocarbon, such as methylene chloride, at preferred temperatures of from −20 to 50° C., especially from −10 to 30° C.

As already mentioned, in the case of the said intermediates it is possible, if necessary or desirable, for protecting groups to be introduced, to be present or to be removed at suitable stages. The person skilled in the art will know which protecting groups can be used for which reactions and compounds of formulae I to XII. In the case of compounds of formula VI that are to be converted into compounds of formula VII, it is advisable to use especially those protecting groups which would not also react during the (Friedel-Crafts-analogous) reaction, that is to say without aryl radicals, such as phenyl radicals. Hydroxy-protecting groups Rₐ and Rₐ′ are especially those which can be selectively introduced and removed, more especially those which are not removed during the conversion of compounds of formula XI. Here it is especially advisable to use hydroxy-protecting groups that do not contain too strongly electronegative substituents, more especially lower alkanoyl, such as acetyl, lower alkoxy-lower alkanoyl, such as methoxyacetyl, or protecting groups of the substituted methyl type, especially lower alkoxymethyl, more especially methoxymethyl (MOM), or lower alkoxy-lower alkoxymethyl, especially 2-methoxyethoxymethyl.

### EXAMPLES

The following Examples serve to illustrate the invention but do not limit the scope thereof.

Abbreviations used:
Celite Celite®, filtration aid based on kieselguhr, Manville Service Corp., USA
conc. concentrated
DMF dimethylformamide
ether diethyl ether
h hour(s)
min minute(s)
NMR nuclear magnetic resonance spectroscopy
PLE pig liver esterase
THF tetrahydrofuran
TLC thin-layer chromatography

The following reaction scheme shows the reactions mentioned in the Examples, the specific radicals being mentioned in the respective Examples:

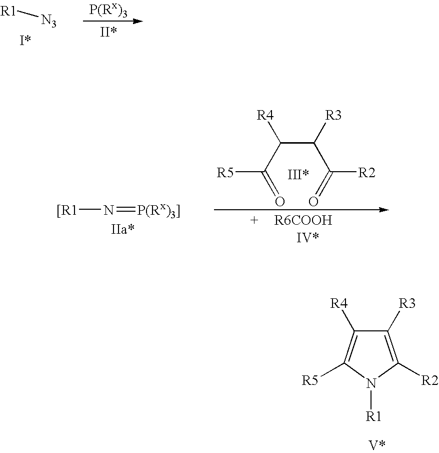

### Example 1

### Preparation of an Atorvastatin Precursor

Substituents in the formulae:
R^{x}=n-butyl (formula II*, IIa*), 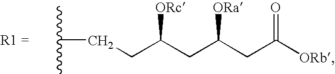
Rc′—Ra′=together isopropylidene, Rb′=ethyl (formula I*, IIa*, V*); 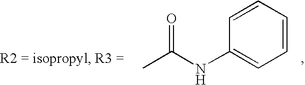
R4=phenyl, R5=4-fluorophenyl (formula III*, V*);
R6=2,4,6-trimethylphenyl (formula IV*).

The azide I*, 1.00 g (3.73 mmol), is dissolved at room temperature in 3 ml of dry toluene, and 0.92 ml (3.73 mmol) of tributylphosphine II* is added. On vigorous stirring, nitrogen begins to evolve. When the evolution of gas has ceased (and TLC monitoring) the mixture is added dropwise to a mixture of diketone III, 1.2 g (2.87 mmol) and 0.61 g (3.73 mmol) of 2,4,6-trimethylbenzoic acid IV* and molecular sieve 3A (Fluka, Buchs, Switzerland) in 6 ml of dry toluene at 60° C. When the reaction is complete (TLC monitoring), the mixture is extracted with 1N sodium hydroxide solution, 1N hydrochloric acid and saturated sodium chloride solution. The product is separated therefrom by column chromatography on silica gel (eluant CH₂Cl₂-ethyl acetate such as 30-0.5 to 3-2). 1.26 g (70%) of pyrrole V are obtained.

¹H-NMR (300 MHz) in CDCl₃ (ppm): 1.06 q (1H; 12.5 Hz); 1.2 t m (3H, 7.5 Hz); 1.31 s (3H); 1.35-1.40 m (1H); 1.37 s (3H); 1.54 d (6H; 7.3 Hz); 1.63-1.74 m (2H); 2.31 dd (1H; 6.2 Hz, 15.3 Hz); 2.49 dd (1H; 7.0 Hz; 15.3 Hz); 3.58 sept. (1H; 7.3 Hz); 3.65-3.75 m (1H); 3.78-3.89 m (1H); 4.02-4.26 m (4H); 6.87-7.20 m (15H).

¹³C-NMR (75.4 MHz) in CDCl₃ (ppm): 14.57; 20.05; 21.95; 22.12; 26.47; 30.27; 36.33; 38.40; 41.61; 60.67; 65.95; 66.69; 99.00; 115.54 d (J_{C,F} 21.3 Hz); 115.57; 119.77; 122.01; 123.67; 126.73; 128.51; 128.83; 128.97; 130.67; 133.55 d (J_{C,F} 8.1 Hz); 134.99; 138.61; 141.64; 162.40 d (J_{C,F} 247.3 Hz); 164.94; 170.90.

The removal of the protecting groups and the further processing of the resulting compound to form the desired atorvastatin can be carried out analogously to the literature (WO89/07598, in this respect incorporated herein by reference).

The diketone starting material of formula III* is known (see WO 89/07598).

The starting material of formula I* is prepared as follows: 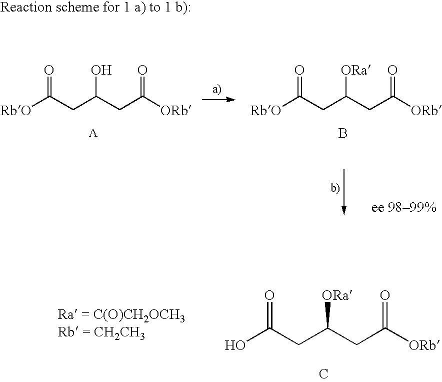
a) Precursor of Formula B, wherein Rb′=ethyl, Ra′=methoxyacetyl (diethyl 3-(methoxy)acetoxyglutaric acid):

50.0 g of diethyl 3-hydroxyglutaric acid A (Fluka, Buchs, Switzerland) are dissolved at 0° C. in 80 ml of dichloromethane; 20.6 ml of pyridine and 22.9 ml of methoxyacetyl chloride are added and the reaction mixture is stirred at room temperature for about 12 h until all the starting material has reacted. The mixture is washed in succession with water, 1N hydrochloric acid, saturated sodium hydrogen carbonate solution and saturated sodium chloride solution. The organic phase is separated off and dried over magnesium sulfate. After evaporation of the organic solvent, a dark-yellow syrup is obtained which is filtered using hexane/ethyl acetate (2:1, v/v) over a small amount of silica gel. After evaporation of the solvent, 65.0 g of NMR-spectroscopically pure methoxy acetate B are obtained.

¹H-NMR (CDCl₃): 1.20 (t, 3H); 2.65 (d, 4H); 3.35 (s, 3H); 3.90 (s, 2H); 4.04 (q, 4H); 5.55 (quin., 1H);

b) Compound of Formula C, wherein Rb′=ethyl, Ra′=methoxyacetyl (monoethyl-3 (R)-3-(methoxy-)acetoxyglutaric acid):

40.0 g of diethyl 3-acetoxymethoxyglutaric acid B are suspended at room temperature in 150 ml of dist. water, and 43 ml of 0.1M phosphate buffer (pH=7) are added. After the addition of 0.4 g of chymotrypsin the mixture is stirred vigorously and maintained at pH=7.8 using a pH meter and pH stat (Metrohm) and 0.5N sodium hydroxide solution. After 18 h a further 0.1 g of chymotrypsin is added and the mixture is stirred until the theoretical amount of hydroxide solution has been consumed. The mixture is then extracted with ethyl acetate (4×). The aqueous phase is adjusted to pH=1 with conc. hydrochloric acid and then extracted with ethyl acetate. Any cloudiness of the organic phase can be removed by filtration over Celite®. The organic phase is further extracted with saturated sodium chloride solution and dried over sodium sulfate. After evaporation of the organic phase, 24.8 g of compound C remain behind.

¹H-NMR (CDCl₃): 1.24 (t, 3H); 2.74 (d, 2H); 2.75 (d, 2H); 3.42 (s, 3H); 3.99 (s, 2H); 4.14 (q, 2H); 5.59 (quin., 1H); 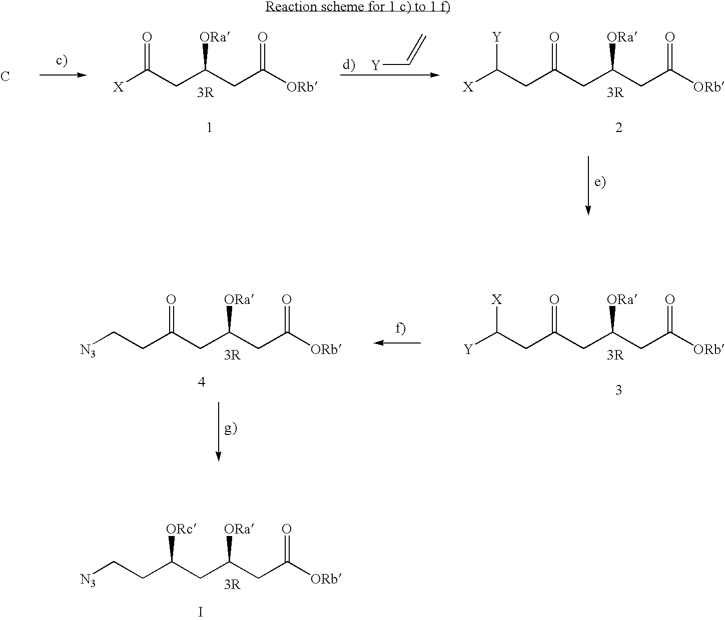
c) Monoethyl ester of (3R)-(methoxy)acetoxyglutaric acid chloride 1, wherein Rb′=ethyl, Ra′=methoxyacetyl, X=chlorine:

21.0 g of monoacid C are dissolved in 100 ml of dry dichloromethane to which 40 μl of dry dimethylformamide have been added, and at 0-5° C. slowly treated with 13.9 g of oxalyl chloride. The mixture is then stirred for about a further 4 h, the temperature of the mixture rising to room temperature. The mixture is then diluted with ethyl acetate and extracted 3× with ice-water, and the organic phase is dried over sodium sulfate. After evaporation of the solvent, 20.9 g of NMR-spectroscopically pure acid chloride 1 remain behind.

¹H-NMR (CDCl₃): 1.20 (t, 3H); 2.04 (s, 3H); 2.67 (m, 2H); 3.32 (m, 2H); 3.36 (s, 3H); 3.95 (s, 2H); 4.09 (q, 2H); 5.52 (m, 1H);

d) 3-R-(Methoxy)acetoxy-7-chloro-5-oxo-heptanoic acid ethyl ester 2, wherein Rb′=ethyl, Ra′=methoxyacetyl, X=chlorine, Y=hydrogen:

20.0 g of acid chloride I are dissolved at 0°-10° C. in 50 ml of dry ethylene chloride and in the course of 20 min added dropwise to 30.0 g of aluminium trichloride in 300 ml of ethylene chloride, a slight rise in temperature being observed. Dry ethylene gas is passed through the resulting suspension, the temperature rising to about 10° C. and the suspension largely passing into solution. When the absorption of gas is complete, the mixture is poured into ice-cold saturated sodium chloride solution, the organic phase is separated off and washed a further 4× with saturated sodium chloride solution. The resulting oil is used further in crude form. Analytically pure material is obtained by chromatography on silica gel (eluant: hexane/ethyl acetate: 2:1, v/v). 13.9 g of chloride 2 are obtained.

¹H-NMR (CDCl₃): 1.25 (t, 3H); 2.70 (m, 2H); 2.91 (m, 4H); 3.41 (s, 3H); 3.72 (t, 2H); 3.97 (s, 2H); 4.13 (q, 2H); 5.62 (m, 1H);

e) 3-R-Hydroxy-7-chloro-5-oxo-heptanoic acid ethyl ester 3, wherein Rb′=ethyl, Ra′=H, X=chlorine, Y=hydrogen:

45.0 g of diester 2 are suspended at room temperature in 500 ml of bi-distilled water and adjusted to pH=6.5 using 0.5N sodium hydrogen carbonate solution. The solution is stirred vigorously; 2.0 ml (500 kU/ml) of technical-grade pig liver esterase (Boehringer) are added and the mixture is maintained at pH=6.5 by means of a pH stat (Metrohm) and 0.5N sodium hydrogen carbonate solution. When the theoretical amount of base has been consumed, extraction is carried out with ethyl acetate. The organic phase is then washed with saturated sodium chloride solution and dried over magnesium sulfate (lipophilic impurities can be removed, if necessary, by extraction with hexane). 29.6 g of pale yellow, oily product 3 are obtained.

¹H-NMR (CDCl₃): 1.22 (t, 3H); 2.49 (d, 2H); 2.65 (m, 2H); 2.91 (t, 2H); 3.70 (t, 2H); 4.13 (q, 2H); 4.46 (m, 1H);

¹³C-NMR (75.4 MHz) in CDCl₃ (ppm): 14.0; 38.1; 40.9; 46.0; 49.1; 60.9; 64.5; 172.1; 206.8.

f) 3-R-Hydroxy-7-azido-5-oxo-heptanoic acid ethyl ester 4, wherein Rb′=ethyl, Ra′=H:

47.8 g of chlorine compound 3 are introduced at 0° C. into 160 ml of DMF, and 15.5 g of sodium azide (Riedel de Haen) are added. With vigorous stirring, the reaction mixture is heated to room temperature (about 14 h). The mixture is then diluted with ethyl acetate and extracted in succession with water, saturated sodium hydrogen carbonate solution and water. The organic phase is dried over magnesium sulfate and concentrated by evaporation. 49.5 g of ketoazide 4 are obtained.

¹H-NMR (CDCl₃): 1.22 (t, 3H); 2.47 (d, 2H); 2.64 (m, 2H); 2.69 (t, 2H); 3.40 (broad OH); 3.50 (t, 2H); 4.11 (q, 2H); 4.44 (m, 1H);

¹³C-NMR (75.4 MHz) in CDCl₃ (ppm): 14.3; 40.9; 42.5; 45.7; 49.0; 60.9; 64.5; 172.0; 207.3.

g) (3R,5R)-7-Azido-3,5-dihydroxy-heptanoic acid ethyl ester Ia* (Rb′=ethyl, Ra′ and Rc′=each H): 0.28 g of ketoazide 4 is dissolved in 2 ml of dry THF. A mixture of 2.5 ml of dry methanol and 9.5 ml of dry THF is prepared under an argon atmosphere at room temperature and 1.4 ml of triethylborane are added. The mixture is stirred for 1 h at room temperature and then cooled to −65° C. The starting material is then added dropwise to the resulting solution in the course of 30 min. At 65° C. a total of 0.054 g of sodium borohydride is then added in portions and stirring is continued for a further 1 h at −65° C. 5% ammonium chloride solution is added and the mixture is brought to room temperature. Extraction with ethyl ester is then carried out. The reaction mixture is brought to room temperature, diluted with ethyl acetate and extracted with 5% ammonium chloride solution. The organic phase is separated off and dried over magnesium sulfate. After removal of the solvent, the residue is evaporated a further 5× with 40 ml of methanol and purified by chromatography over silica gel. 0.20 g of oily diol Ia* are obtained: ¹H-NMR (D₂O): 1.25 (t, 3H); 1.56 (m, 2H); 1.68 (m, 2H); 2.46 (d, 2H); 3.34 (m, 2H); 3.97 (m, 1H); 4.14 (q, 2H); 4.25 (m, 1H).

(Alternatively, the residue can be taken up in THF at 0° C. and cautiously oxidised at 0° C. with from 1 to 1.2 equivalents of 30% H₂O₂. After extraction with ethyl acetate and drying over magnesium sulfate, the product can be repeatedly concentrated by evaporation with methanol and/or purified by chromatography).

g*) (Conversion Subsequent to (g)) (3R,5R)-7-azido-3,5-(2′,2′-ethylidene-dioxy)heptanoic acid ethyl ester I* (Rb′=ethyl, Ra′, Rc′ together=ethylidene):

1.3 g of diol Ia* are dissolved in 1.3 ml of acetaldehyde diethyl acetal in 10 ml of THF and at room temperature 10.0 mg of para-toluenesulfonic acid are added. When the reaction is complete (TLC monitoring), the mixture is neutralised with sodium hydrogen carbonate and filtered, the solvent is evaporated and the residue is purified by chromatography on silica gel. 0.9 g of colourless oil is obtained.

¹H-NMR (300 MHz) in CDCl₃ (ppm): 1.23 t (3H, 7.0 Hz); 1.28 d (2H; 5.3 Hz); 1.30 m (1H); 1.58 dt (1H; 2.3 Hz, 12.9 Hz); 1.73 m (2H); 2.38 dd (1H; 6.2 Hz, 15.5 Hz); 2.58 dd (1H; 7.0 Hz; 15.5 Hz); 3.39 m (2H); 3.72 m (1H); 4.05 m (1H); 4.12 q (1H; 7.0 Hz); 4.68 q (1H, 5.3 Hz).

¹³C-NMR (75.4 MHz) in CDCl₃ (ppm): 14.53; 21.31; 35.36; 36.54; 41.22; 47.67; 60.82; 72.77; 73.07; 98.90; 115.11; 170.67.

A different compound of formula I* having isopropylidene in place of ethylidene Ra′ and Rc′ is obtained analogously:

g**) (Conversion Subsequent to (g)) (3R,5R)-7-azido-3,5-(2′,2′-isopropylidene-dioxy)heptanoic acid ethyl ester I* (Rb′=ethyl, Ra′, Rc′=together isopropylidene): 0.50 g of compound Ia* is dissolved in 1 ml of absolute THF, and at room temperature 0.25 g of dimethoxypropane and 0.01 g of toluenesulfonic acid are added. After 2.5 h, the reaction mixture is diluted with ethyl acetate and extracted in succession with saturated sodium chloride solution, saturated sodium hydrogen carbonate solution and saturated sodium chloride solution. After removal of the solvent, 0.50 g of product I* is obtained: ¹H-NMR (CDCl₃): 1.19 (t, 1H); 1.25 (t, 3H); 1.36 (s, 3H); 1.45 (s, 3H); 1.58 (dt, 1H); 1.70 (m, 2H); 2.32 (m, 2H); 2.51 (m, 2H); 3.38 (m, 2H); 4.00 (m, 1H); 4.14 (dq, 2H); 4.31 (m, 1H).

### Example 2

### Preparation of an Atorvastatin Precursor (Variant)

Substituents in the formulae: 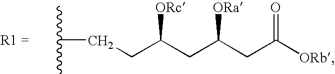
Rc′—Ra′=together ethylidene, Rb′=ethyl (formula II*, IIa*, V*);
R^{x}=n-butyl (formula II*, IIa*), 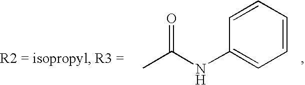
R4=phenyl, R5=4-fluorophenyl (formula III*, V*);
R6=2,4,6-triisopropylphenyl (formula IV*).

According to the process of Example 1, from 0.90 g of azide I* and 1.10 g of diketone III* in the presence of 0.87 g of tri-isopropylbenzoic acid there are obtained 1.08 g (68%) of pyrrole V having the substituents mentioned at the beginning.

¹H-NMR (300 MHz) in CDCl₃ (ppm): 1.12-1.40 m (8H); 1.54 dd (6H; 7.1 Hz, 7.1 Hz); 1.66-1.78 m (2H); 2.33 dd (1H; 6.2 Hz, 15.3 Hz); 2.54 dd (1H; 7.0 Hz; 15.3 Hz); 3.43 m (1H); 3.53 sept. (1H; 7.1 Hz); 3.96 m (2H); 3.96-4.15 m (3H); 4.52 q (1H, 7.1 Hz), 6.88-7.19 m (15H).

¹³C-NMR (75.4 MHz) in CDCl₃ (ppm): 15.29; 21.96; 22.70; 22.98; 35.94; 38.60; 41.84; 61.59; 73.40; 73.80; 99.49; 116.17 d (J_{C,F} 21.3 Hz); 116.53; 120.50; 122.82; 124.42; 127.49; 129.25; 129.56; 129.71; 131.41; 134.10 d (J_{C,F} 8.1 Hz); 135.60; 139.35; 142.30; 164.10 d (J_{C,F} 247.3 Hz); 165.66; 171.42.

### Example 3

### 1-(n-Hexyl)-5-(4-fluorophenyl)-2-isopropyl-4-phenyl-3-phenylaminocarbonyl-pyrrole

Substituents in the formulae:
R^{x}=n-butyl (formula II*, IIa*),
R1=n-hexyl (formula I*, IIa*, V*); 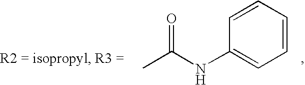
R4=phenyl, R5=4-fluorophenyl (formula III, V*);
R6=2,4,6-triisopropylphenyl (formula IV*).

Analogously to the process of Example 1, from 0.46 g of azide I* and 1.35 g of diketone III* there is obtained 0.92 g (59%) of pyrrole V*, having the substituents mentioned at the beginning of this Example:

¹H-NMR (300 MHz) in CDCl₃ (ppm): 0.85 t (3H; 6.5 Hz); 1.13-1.24 m (6H); 1.54-1.60 m (8H); 3.55 sept. (1H; 7.3 Hz); 3.77-3.86 m (2H); 6.87-7.22 m (15H).

¹³C-NMR (75.4 MHz) in CDCl₃ (ppm): 13.88; 21.71; 22.35; 26.26; 26.33; 31.04; 31.50; 44.66; 115.00 d (J_{C,F} 21.3 Hz); 115.03; 119.37; 121.50; 123.25; 126.34; 128.13; 128.29 d (J_{C,F} 8.1 Hz); 128.45; 128.68; 130.36; 133.02 d (J_{C,F} 6.0 Hz); 134.60; 138.30; 141.20; 162.02 d (J_{C,F} 247.3 Hz); 164.56; 170.79.

### Example 4

### 1-(n-Hexyl)-2-methyl-5-phenyl-pyrrole

Substituents in the formulae:
R^{x}=n-butyl (formula II*, IIa*);
R1=n-hexyl (formula I*, IIa*, V*);
R2=methyl, R3=H, R4=H, R5=phenyl, R6=2,4,6-trimethylphenyl (formula III*, V*)
R6=2,4,6-triisopropylphenyl (formula IV*).

Analogously to the process of Example 1, from 0.86 g of azide I* and 1.0 g of diketone III* there are obtained 1.10 g (81%) of pyrrole V*, having the substituents mentioned at the beginning of this Example:

¹H-NMR (300 MHz) in CDCl₃ (ppm): 0.74 t (3H; 6.5 Hz); 1.03-1.15 m (6H); 1.40-1.52 m (2H); 2.32 d (3H. 0.9 Hz); 5.85 dq (1H. 0.9 Hz, 3.5 Hz); 5.98 d (1H, 3.5 Hz); 7.14-7.28 m (5H).

¹³C-NMR (75.4 MHz) in CDCl₃ (ppm): 12.79; 13.99; 22.51; 26.36; 31.08; 31.27; 44.18; 106.60; 107.66; 126.43; 128.14; 128.88; 129.52; 133.74; 134.38.

### Example 5

Substituents in the formulae:
R^{x}=n-butyl (formulae II*, IIa*); 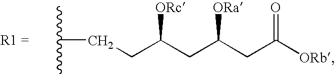
Rc′—Ra′=cyclohexylidene, Rb=ethyl;
R2=isopropyl, 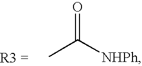
R4=phenyl, R5=4-fluorophenyl (formulae I*, IIa*, V*);
R6=2,4,6-triisopropylphenyl (formula IV*).

Analogously to the process of Example 1, from 2.25 g of azide I* and 2.41 g of diketone III* in the presence of 1.80 g of 2,4,6-tri-isopropylbenzoic acid there is obtained 0.35 g of pyrrole V*.

¹H-NMR (300 MHz) in CDCl₃ (ppm): 1.10 q (1H; 12.5 Hz); 1.26 t (3H, 7.5 Hz); 1.37 s (3H); 1.36-1.58 m (9H); 1.60-1.90 m (4H); 2.32 dd (1H; 6.2 Hz, 15.3 Hz); 2.48 dd (1H; 7.0 Hz; 15.3 Hz); 3.62 sept. (1H; 7.3 Hz); 3.67-3.77 m (1H); 3.78-3.89 m (1H); 4.08-4.31 m (4H); 6.86-7.20 m (15H).

¹³C-NMR (75.4 MHz) in CDCl₃ (ppm): 14.27; 21.63; 21.73; 22.51; 25.73; 26.10; 28.47; 36.32; 38.20; 38.70; 41.05; 41.45; 60.46; 64.81; 65.50; 98.73; 115.22 d (J_{C,F} 21.3 Hz); 115.18; 119.43; 121.65; 123.33; 126.41; 128.12; 128.19; 128.37; 128.50; 128.69; 130.36; 133.01 d (J_{C,F} 8.1 Hz); 134.54; 138.27; 141.36; 162.40 d (J_{C,F} 247.3 Hz); 164.94; 170.90.

The starting material I* for this reaction is obtained from diol I in accordance with the following procedure:

2.0 g of diol I (see Example 1 g)) are dissolved at room temperature in 5 ml of THF, and stirred with 2 ml of cyclohexanedimethylacetal and 10 mg of para-toluenesulfonic acid until all the diol has been converted (about 4-5 h). After neutralisation with sodium hydrogen carbonate, filtration and evaporation of the solvent there remains behind an oil which is purified by chromatography on silica gel using hexane/ethyl acetate mixtures as eluant. 2.4 g of acetal I* are obtained.

¹H-NMR (300 MHz) in CDCl₃ (ppm): 1.20-1.60 m (13H); 1.68-1.74 m (2H); 1.82-1.98 m (2H); 2.37 dd (1H; 5.6 Hz, 15.2 Hz); 2.52 dd (1H; 7.4 Hz; 15.2 Hz); 3.42 t (2H; 6.4 Hz); 3.96-4.06 m (1H); 4.14 q (2H; 7.0 Hz); 4.32 m (1H).

¹³C-NMR (75.4 MHz) in CDCl₃ (ppm): 14.62; 22.78; 22.96; 26.10; 28.87; 33.96; 37.10; 39.14; 41.92; 47.88; 60.77; 65.07; 65.32; 99.23; 171.04.

### Example 6

Substituents in the formulae:
R^{x}=n-butyl (formulae II*, IIa);
R1=n-hexyl, R2=isopropyl, R3=C(O)O-ethyl, R4=phenyl, R5=4-fluorophenyl (formulae III*, V*);
R6=2,4,6-triisopropylphenyl (formula IV*).

Analogously to the process of Example 5, from 0.5 g of azide I* and 1.2 g of diketone III* there is obtained 0.30 g (21%) of pyrrole V*.

¹H-NMR (300 MHz) in CDCl₃ (ppm): 0.83 t (3H. 7.5 Hz); 0.94 t (3H; 7.5 Hz); 1.10-1.35 m (6H); 1.52 d (6H. 7.0 Hz); 3.44 sept (1H, 7.0 Hz); 3.72-3.76 m (2H); 4.02 q (2H. 7.5 Hz); 6.91-7.17 m (9H).

¹³C-NMR (75.4 MHz) in CDCl₃ (ppm): 13.67; 13.86; 21.35; 22.34; 26.21; 26.29; 31.08; 31.45; 44.62; 59.50; 111.15; 114.97 d (J_{C,F} 21.3 Hz); 125.28; 126.92; 129.03; 130.11; 133.08 d (J_{C,F} 8.1 Hz); 135.87; 142.10; 161.85 d (J_{C,F} 247.3 Hz); 166.37.

a) Diketone III* is Obtained as Follows:

10.0 g of 2-bromo-1-(4-fluorophenyl)-1-oxo-2-phenylethane and 7.7 g of isobutyrylacetic acid ethyl ester (Fluka) are dissolved at 0° C. in 80 ml of dry DMF, and 6.7 g of potassium carbonate are added. The mixture is allowed to rise to room temperature, the starting materials reacting completely. The reaction mixture is then filtered, diluted with ethyl acetate and washed in succession with water and saturated sodium chloride solution. The residue obtained after subseqent evaporation of the solvent is purified by chromatography on silica gel using hexane/ethyl acetate mixtures. 8.0 g of diketone III* are obtained in the form of a diastereoisomeric mixture (about 1:1), which is reacted further without further purification.

b) 1-(4-Fluorophenyl)-2-phenylethan-1-one:

(see also H. Buu-Hoi et al., Recl. Tav. Chim. Pays-Bas 1949, 68, 781; Organikum, 16th edition, VEB Deutscher Verlag der Wissenschaften, Berlin 1986, p. 325 f.)

160 g (1.2 eq.) of powdered aluminium chloride are added to 500 ml (about 5 eq.) of fluorobenzene and, with stirring and cooling with ice-water, 138 ml of phenacetyl chloride 1 (1.05 eq.) are added dropwise thereto in such a manner that an internal temperature of 20° C. is not exceeded. 15 min after the end of the addition, the mixture is heated at 50° C. for 5 h and the resulting deep-green solution is kept at room temperature for a further 9 h. Hydrolysis is effected by pouring the reaction mixture onto 500 g of crushed ice and extracting the resulting suspension with 300 ml of 2N HCl. The organic phase is then cautiously washed with sodium hydrogen carbonate solution and saturated sodium chloride solution and dried over sodium sulfate. After removal of the solvent, the solid that remains behind is washed intensively with hexane. 193 g (193 mmol), 90%, of title compound 2 are obtained in the form of a white solid: m.p. 82° C., ¹H-NMR (CDCl₃=7.26 ppm): 4.26 (s, 2H, CH₂); 7.12 (m, 2H, ar); 7.30 (m, 5H, ar); 8.04 (m, 2H, ar). ¹³C-NMR (CDCl₃=77.4 ppm): 196.2, 167.7, 164.3, 134.7, 133.3, 131.6, 131.5, 129.7, 129.0, 127.2, 116.1, 115.8, 45.7.

c) 2-Bromo-1-(4-fluorophenyl)-1-oxo-2-phenylethane:

(see also P. J. Roy et al, Heterocycles 45(11), 2239-46 (1997) in respect of the reaction mechanism and CAS 88675-31-4 in respect of the compound)

273.4 g (1.28 mol) of 1-(4-fluorophenyl)-2-phenylethane 2 are introduced into 2.9 litres of chloroform; 7 ml of a 30% solution of hydrobromic acid in glacial acetic acid are added and 66 ml (1 eq.) of bromine dissolved in 250 ml of chloroform are added dropwise in such a manner that the bromine immediately reacts away. At the end of the reaction, a slight bromine coloration should remain. 10% sodium sulfite solution is added to the reaction mixture, which is then washed with water, sodium hydrogen carbonate solution and saturated sodium chloride solution and dried over sodium sulfate. 375 g (1.28 mol) of pure title compound I are obtained in the form of a reddish brown oil which tends to crystallise at low temperature. M.p.: 46° C., ¹H-NMR (CDCl₃=7.26 ppm): 6.34 (s, H. CHBr), 7.12 (m, 2H, ar), 7.35 (m, 3H, ar), 7.51 (m, 2H, ar), 8.02 (m, 2H, ar); ¹³C-NMR (CDCl₃=77.3 ppm): 189.8, 167.9, 164.5, 136.0, 132.2, 132.1, 129.5, 129.3, 129.3, 116.4, 116.1, 51.2.

### Example 7

Substituents in the formulae:
R^{x}=n-butyl (formulae III, IIa*),
R1=n-hexyl, R2=isopropyl, R3=H, R4=phenyl, R5=4-fluorophenyl (formulae I*, IIa*, V*);
R6=2,4,6-triisopropylphenyl (formula IV*).

Analogously to the process of Example 5, from 0.76 g of azide I* and 1.5 g of diketone III* there are obtained at 60° C. 1.47 g (81%) of pyrrole V*.

¹H-NMR (300 MHz) in CDCl₃ (ppm): 0.87 t (3H. 7.5 Hz); 1.12-1.34 m (6H); 1.40 d (6H, 7.1 Hz); 1.44-1.54 m (2H); 3.01 sept (1H. 7.0 Hz); 3.72-3.76 m (2H); 6.25 s (1H); 6.91-7.17 m (9H).

¹³C-NMR (75.4 MHz) in CDCl₃ (ppm): 14.30; 22.78; 24.10; 26.23; 26.77; 31.53; 31.85; 44.18; 59.50; 103.60; 115.75 d (J_{C,F} 21.3 Hz); 122.12; 124.96; 127.71; 128.17; 130.22 d (J_{C,F} 8.1 Hz); 133.32 d (J_{C,F} 8.1 Hz); 136.85; 140.30; 164.04 d (J_{C,F} 247.3 Hz); 166.37.

The diketone III* is obtained as follows (analogously to: L. Nilsson, C. Rappe, Acta. Scand. 30 B 1976, 10, 1000):

From 1.96 g of 2-bromo-1-(4-fluorophenyl)-1-oxo-2-phenylethane and 1.60 g of 4-(3-methyl-1-buten-2-yl)pyrrolidine (see W. White, H. Weingarten, J. Org. Chem. 17, 32, 213) there is obtained 0.31 g of diketone III* after purification by chromatography on silica gel using hexane/ethyl acetate mixtures as eluant.

¹H-NMR (300 MHz) in CDCl₃ (ppm): 1.09 d (3H, 6.8 Hz); 1.13 d (3H, 6.8 Hz); 2.64 sept (1H, 6.8 Hz); 2.79 dd (1H, 3.8 Hz, 17.8 Hz); 3.63 dd (1H. 10.0 Hz, 17.8 Hz); 5.07 dd (1H, 3.8 Hz, 10.0 Hz); 6.97-7.05 m (2H); 7.14-7.31 m (5H); 7.96-9.05 m (2H).

¹³C-NMR (75.4 MHz) in CDCl₃ (ppm): 18.49; 18.51; 41.12; 45.52; 48.95; 115.74 d (J_{C,F} 21.3 Hz); 127.56; 128.23; 129.38; 131.60; 131.72; 133.03 d (J_{C,F} 8.1 Hz); 138.69; 165.63 d (J_{C,F} 247.3 Hz); 197.48; 212.71.

### Example 8

Substituents in the formulae:
R^{x}=n-butyl (formula II*, IIa*);
R1=n-hexyl, R2=isopropyl, R3=H, R4=H, R5=phenyl (formulae I*, IIa*, V*);
R6=2,4,6-triisopropylphenyl (formula IV*).

Analogously to the process of Example 5, from 0.50 g of azide I* and 0.66 g of diketone III* there is obtained at 60° C. 0.65 g (75%) of pyrrole V*.

¹H-NMR (300 MHz) in CDCl₃ (ppm): 0.89 t (3H, 7.5 Hz); 1.17-1.30 m (6H); 1.40 d (6H, 7.1 Hz); 1.50-1.62 m (2H); 3.03 sept (1H, 7.0 Hz); 3.87-4.01 m (2H); 6.08 d (1H. 3.5 Hz); 6.25 d (1H, 3.5 Hz); 7.30-7.48 m (5H).

¹³C-NMR (75.4 MHz) in CDCl₃ (ppm): 14.35; 22.85; 24.19; 26.36; 26.87; 31.62; 31.92; 44.33; 103.36; 108.35; 126.77; 128.48; 128.56; 133.74; 134.85; 141.37.

The diketone III* is obtained as follows (analogously to: L. Nilsson, C. Rappe, Acta. Scand. 30 B 1976, 10, 1000):

From 5.40 g of phenacyl bromide and 5.00 g of 4-(3-methyl-1-buten-2-yl)pyrrolidine (W. White, H. Weingarten, J. Org. Chem. 1967, 32, 213) there is obtained 0.66 g of diketone III* after purification by chromatography on silica gel using hexanelmethylene chloride mixtures as eluant.

¹H-NMR (300 MHz) in CDCl₃ (ppm): 1.15 d (3H, 6.8 Hz); 2.71 sept (1H, 6.8 Hz); 2.79 t (2H, 6.5 Hz); 3.25 t (2H, 6.5 Hz); 7.38-7.55 m (3H); 7.93-8.05 m (2H).

¹³C-NMR (75.4 MHz) in CDCl₃ (ppm): 18.70; 32.71; 34.32; 41.29; 128.20; 128.72; 133.22; 136.95; 198.74; 213.19.

### Example 9

Substituents in the formulae:
R^{x}=n-butyl (formulae II*, IIa*);
R1=n-hexyl, R2=methyl, R3=tert-butoxycarbonyl, R4=methyl, R5=phenyl (formulae I*, IIa*, V*);
R6=2,4,6-triisopropylphenyl (formula IV*).

Analogously to the process of Example 5, from 1.15 g of azide I* and 2.20 g of diketone III* there are obtained at 60° C. 1.74 g (65%) of pyrrole V*.

¹H-NMR (300 MHz) in CDCl₃ (ppm): 0.81 t (3H, 7.5 Hz); 1.07-1.22 m (6H); 1.59-1.63 m (2H); 1.60 s (9H); 2.11 s (3H); 2.57 s (3H); 3.67-3.74 m (2H); 7.22-7.44 m (5H).

¹³C-NMR (75.4 MHz) in CDCl₃ (ppm): 12.22; 12.37; 14.26; 22.71; 26.57; 29.02; 30.99; 31.45; 44.31; 117.89; 121.01; 127.69; 128.43; 130.76; 131.38; 134.93; 144.89; 166.00.

The diketone III* is obtained as follows (analogously to: F. Stauffer, R. Neier, Org. Lett. 2000, 2(23), 3535):

From 1.94 ml of bromopropiophenone and 1.90 ml of tert-butyl acetoacetate there are obtained, after purification by chromatography on silica gel using hexane/ethyl acetate mixtures as eluant, 2.14 g of diketone III* in the form of a mixture of two diastereoisomers (about 6:4) which are used further without further purification.

¹H-NMR (300 MHz) in CDCl₃ (ppm): main isomer 1.18 d (3H. 6.8 Hz); 1.50 a (9H); 2.26 s (3H); 3.98-4.08 m (1H); 7.39-7.56 m (3H); 7.93-8.05 m (2H); 7.96-8.05 m (2H).

¹³C-NMR (75.4 MHz) in CDCl₃ (ppm): main isomer: 16.23; 28.26; 29.72; 64.25; 128.25; 128.70; 133.26; 135.85; 167.68; 201.84; 202.34.

### Example 10

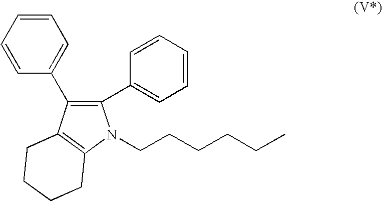

(Substituents in the formulae:
R^{x}=butyl (formulae II* and IIa*);
R1=n-hexyl, R2=phenyl, R3=phenyl, R4, R5 together=n-butylidene (formulae I*, IIa*, VI);
R6=2,4,6-triisopropyl (formula IV*).)

Analogously to the process of Example 5, from 0.47 g of azide I* and 0.90 g of diketone III* there is obtained at 60° C. 0.76 g (69%) of pyrrole V*.

¹H-NMR (300 MHz) in CDCl₃ (ppm): 0.73 t (3H. 7.5 Hz); 1.02-1.14 m (6H); 1.40-152 m (2H); 1.65-1.74 m (2H); 1.80-1.88 m (2H); 2.56-2.60 m (2H); 3.59-3.65 m (4H); 6.94-7.21 m (10H).

¹³C-NMR (75.4 MHz) in CDCl₃ (ppm): 14.34; 22.84; 22.99; 23.45; 23.87; 24.32; 24.37; 24.55; 26.81; 31.59; 44.24; 116.38; 120.87; 124.88; 127.06; 127.87; 128.38; 129.82; 131.47; 133.86; 136.58; 144.93.

The diketone III is obtained as follows (analogously to: L. Nilsson, C. Rappe, Acta. Scand. 30 B 1976, 10, 1000);

From 2.5 g of desyl bromide (α-chlorodeoxybenzoin) and 6.0 ml of morpholinocyclohexene (Fluka) there are obtained, after purification by chromatography on silica gel using hexane/ethyl acetate mixtures as eluant, 1.14 g of diketone III* in the form of a mixture of two diastereoisomers (about 6:4), which are used further without further purification.

¹H-NMR (300 MHz) in CDCl₃ (ppm): 1.20-2.60 m (8H); 3.04-3.12 m (0.4H); 3.44-3.60 m (0.6H); 4.73 d (0.6H, 10.2 Hz); 5.19 d (0.4H. 7.6 Hz); 7.16-7.46 m (2H); 7.93-8.02 m (2H).

¹³C-NMR (75.4 MHz) in CDCl₃ (ppm): main isomer: 25.79; 28.74; 32.51; 42.57; 54.02; 55.11; 128.58; 128.81; 128.83; 129.05; 129.19; 132.71; 136.73; 137.81; 199.31; 211.82.

### Example 11

Substituents in the formulae:
R^{x}=n-butyl (formulae II*, IIa*), 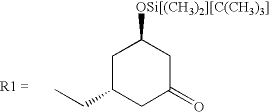
R2=methyl, R3=H, R4=H, R5=phenyl (formulae I*, IIa*, V*);
R6=2,4,6-triisopropylphenyl.

Analogously to the process of Example 4, from 0.30 g of azide I* and 0.16 g of diketone III* (Lancaster) there is obtained 0.10 g of pyrrole V*.

¹H-NMR (300 MHz) in CDCl₃ (ppm): 0.02 (s, 3H); 0.03 (s, 3H); 0.84 (s, 3H), 1.20-1.28 (m, 2H); 1.58-1.84 (m, 2H); 1.40-1.52 (m, 2H); 2.31 (s, 3H); 2.39-2.54 (m, 2H); 4.00-4.21 (m, 3H); 3.38-4.40 (m, 1H); 5.98 (d, 1H, 3.5 Hz); 6.06 (d, 1H, 3.5 Hz); 7.20-7.34 (m, 5H).

¹³C-NMR (75.4 MHz) in CDCl₃ (ppm): 0.00; 17.62; 22.79; 30.55; 41.17; 44.45; 44.01; 44.88; 68.19; 77.97; 111.82; 113.27; 131.48; 133.37; 134.79; 138.13; 138.91; 174.38.

The starting material I* for this reaction is obtained as follows:

15 ml of 1N sodium hydroxide solution are added to 3.60 g of diol Ia* from Example 1 g) (Rc′=Ra′=H, Rb′=ethyl) in 80 ml of ethanol at room temperature and the mixture is stirred until the ester has completely hydrolysed. The mixture is adjusted to pH=2 with 1N sulfuric acid and extracted with diethyl ether. The organic phase is dried over sodium sulfate and evaporated. The residue (2.85 g) is taken up in 3 ml of methylene chloride, and 3.0 g of aluminium chloride (activity stage 1) are added. After 3 days at room temperature, the mixture is filtered and concentrated by evaporation, and the residue is chromatographed (eluant: methylene chloride/ethyl acetate). 1.5 g of lactone are obtained.

This is dissolved at 0° C. in 10 ml of methylene chloride, and 0.95 ml of 2,6-dimethylpyridine and 1.9 ml of tert-butyidimethylsilyl trifluoromethanesulfonate dissolved in 3 ml of methylene chloride are added in succession thereto. When the reaction is complete, extraction is carried out with sodium chloride solution. Evaporation of the solvent leaves a residue which is purified on silica gel (eluant: hexane/ethyl acetate 10:7, v/v). 0.69 g of silylated lactone I* is obtained.

¹H-NMR (300 MHz) in CDCl₃ (ppm): 0.00 (s, 6H); 0.81 (s, 9H); 1.64 (t, 1H; 11.4 Hz); 1.72-1.89 (m, 3H); 2.42-2.58 (m, 2H); 3.43 (t, 3H, 7.3 Hz); 4.21-4.24 (m, 1H); 4.68-4.72 (m, 1H).

¹³C-NMR (75.4 MHz) in CDCl₃ (ppm): 0.00; 22.83; 30.55; 39.75; 41.31; 44.11; 52.04; 66.28; 77.75; 174.31.

1. A process for the preparation of a pyrrole of formula V 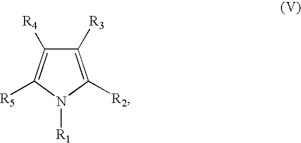
wherein R₁ is an organic substituent and
R₂, R₃, R₄ and R₅ are each independently of the others hydrogen or an inorganic or organic substituent bonded by way of a carbon atom or hetero atom belonging to the R₂, R₃, R₄ or R₅ radical, or a pair or pairs of those radicals may form a bridge bonded by way of carbon and/or hetero atoms, wherein an iminophosphorane of formula IIa
R₁—N═(PR^{x})₃ (IIa),
wherein
R₁ is as defined for compounds of formula V and
R^{x} is unsubstituted or substituted alkyl, unsubstituted or substituted aryl, unsubstituted or substituted alkoxy or unsubstituted or substituted aryloxy,
is reacted with a dioxo compound of formula III 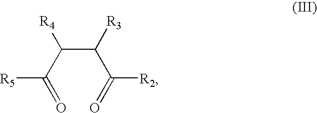
wherein the radicals R₂, R₃, R₄ and R₅ are as defined for compounds of formula V, in the presence of an acid,
functional groups in the starting materials being, if necessary, in protected form and any protecting groups being removed, if necessary, at suitable stages.

2. A process according to claim 1, wherein, in addition, the iminophosphorane of formula IIa is obtained beforehand by reaction of an azide of formula I
R₁—N₃ (I),
wherein R^{x} is as defined for compounds of formula V, with a phosphorus(III) compound of formula II
P(R^{x})₃ (II),
wherein R^{x} is unsubstituted or substituted alkyl, unsubstituted or substituted aryl, unsubstituted or substituted alkoxy or unsubstituted or substituted aryloxy,
functional groups in the starting materials being, if necessary, in protected form and any protecting groups being removed, if necessary, at suitable stages.

3. A process according to claim 1 for the preparation of a pyrrole of formula V wherein
R₁ is unsubstituted or substituted alkyl or unsubstituted or substituted aryl, and
R₂, R₃, R₄ and R₅ are each independently of the others hydrogen, unsubstituted or substituted alkyl, unsubstituted or substituted alkyl-lower alkoxycarbonyl or unsubstituted or substituted aryl, and
R^{x} in the iminophosphorane of formula IIa is alkyl or aryl,
wherein suitably substituted starting materials are used.

4. A process according to claim 1 for the preparation of a pyrrole of formula V wherein
R₁ is lower alkyl or a radical of sub-formula IA or sub-formula IB 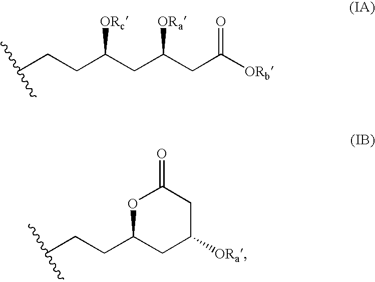
wherein Rₐ′ and R_{c}′ are each independently of the other hydrogen or a hydroxy-protecting group, or Rₐ′ and R_{c}′ together are a bridging hydroxy-protecting group; and R_{b}′ in formula IA is a carboxy-protecting group;
R₂, R₃, R₄ and R₅ are each independently of the others hydrogen, lower alkyl, lower alkoxycarbonyl, phenylaminocarbonyl, phenyl, naphthyl or fluorophenyl, and
R^{x} in the iminophosphorane of formula IIa is lower alkyl or phenyl,
wherein suitably substituted starting materials are used.

5. A process according to claim 4 for the preparation of a pyrrole of formula V wherein
R₁ is lower alkyl or a radical of sub-formula IA shown in claim 4 wherein R_{c}′ and Rₐ′ together are lower alkylidene, and R_{b}′ is lower alkyl, or a radical of sub-formula IB shown in claim 4 wherein Rₐ′ is a hydroxy-protecting group
R₂, R₃, R₄ and R₅ are each independently of the others hydrogen, lower alkyl, lower alkoxycarbonyl, C₆-C₁₄arylaminocarbonyl, C₆-C₁₄aryl or halo-C₆-C₁₄aryl, and
R^{x} in the iminophosphorane of formula IIa is lower alkyl or phenyl,
wherein suitably substituted starting materials are used.

6. A process according to claim 1, wherein the reaction of the iminophosphorane of formula IIa with the dioxo compound of formula III is carried out by combining an iminophosphorane of formula IIa with a mixture of a 1,4-dioxo compound of formula III and an acid in an aprotic solvent or solvent mixture, the reaction being carried out in the presence of reagents that bind the water of reaction formed, the molar ratio of phosphorane imine IIa to dioxo compound III and acid IV being about from 1 to 1.5:1, and the reaction being carried out at temperatures of from room temperature to 110° C.

7. A process according to claim 2, wherein the preparation of the iminophosphorane of formula IIa from an azide of formula I is carried out by reacting an azide of formula I in a dry organic solvent temperatures of from −20° C. to the reflux temperature, with from 1 to 1.5 equivalents of the compound of formula II, it also being possible for the phosphorus(III) compound of formula II to be bound to a polymeric carrier.

8. An iminophosphorane of formula IIa
R₁—N═(PR^{x})₃ (IIa)
wherein R₁ is a radical of sub-formula IA 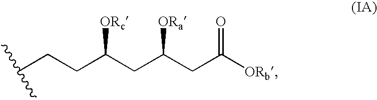
wherein Rₐ′ and R_{c}′ are each independently of the other hydrogen or a hydroxy-protecting group, or Rₐ′ and R_{c}′ together are a bridging hydroxy-protecting group; and R_{b}′ is a carboxy-protecting group; and
R^{x} is unsubstituted or substituted alkyl, unsubstituted or substituted aryl, unsubstituted or substituted alkoxy or unsubstituted or substituted aryloxy.

9. A process for the preparation of atorvastatin, which includes a preparation process according to claim 1, wherein, in the pyrrole of formula V 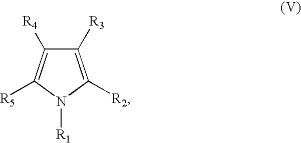
R₁ is a radical of formula IA 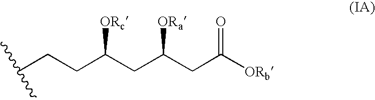
wherein Rₐ′ and R_{c}′ are each independently of the other hydrogen or a hydroxy-protecting group, or Rₐ′ and R_{c}′ together are a bridging hydroxy-protecting group; and R_{b}′ is a carboxy-protecting group;
R₂ is isopropyl,
R₃ is phenylaminocarbonyl,
R₄ is phenyl and
R₅ is 4-fluorophenyl;
wherein suitably substituted starting materials are used, the process optionally including also the removal of protecting groups and/or cleavage of a lactone ring.

10. A process according to claim 6, wherein the acid employed is a moderately acidic ion exchanger, a moderately acidic inorganic acid, an organic acid, or a mixture of such acids, the reaction being carried out in the presence of reagents that bind the water of reaction formed, the molar ratio of phosphorane imine IIa to dioxo compound III and acid IV being about from 1 to 1.5:1, the reaction being carried out at temperatures of from 40° C. to 70° C.

11. A process according to claim 10, wherein the moderately acidic inorganic acid is phosphoric acid, and the organic acid is an organic phosphoric acid derivative or a carboxylic acid, or a mixture of said inorganic and organic acids.
